(19) United States
(12) Patent Application Publication
(10) Pub. No.: US 2021/0231673 A1
(43) Pub. Date: Jul. 29, 2021

(54) METHODS AND COMPOSITIONS FOR DETECTION AND QUANTIFICATION OF SMALL MOLECULES AND OTHER ANALYTES

(71) Applicant: President and Fellows of Harvard College, Cambridge, MA (US)

(72) Inventors: David R. Walt, Boston, MA (US); Xu Wang, Somerville, MA (US)

(21) Appl. No.: 17/267,870

(22) PCT Filed: Aug. 13, 2019
(86) PCT No.: PCT/US2019/046254

§ 371 Date: Feb. 11, 2021

Related U.S. Application Data
(60) Provisional application No. 62/765,069, filed on Aug. 17, 2018.

(51) Int. Cl. G01N 33/68; C12N 9/38; G01N 33/58; G01N 33/532

(52) U.S. Cl. CPC G01N 33/68; C12N 9/2471; C12Y 302/01023; G01N 33/532; G01N 33/58

(57) ABSTRACT

The invention provides high-sensitivity methods for detection and quantification of target analytes (e.g., small molecule target analytes) in samples (e.g., biological or environmental samples). The methods can be multiplexed to allow simultaneous detection and quantification of multiple target analytes, including small molecules and other analytes (e.g., nucleic acids and proteins), that are contained in the same sample. The invention also provides related compositions and kits.

### RELATED APPLICATIONS

The instant application claims priority to U.S. Provisional Application No. 62/765,069, filed on Aug. 17, 2018, the entire contents of which are expressly incorporated herein by reference.

### FIELD OF THE INVENTION

The invention relates to methods and compositions for detection and quantification of analytes (e.g., small molecule analytes).

### BACKGROUND OF THE INVENTION

Immunological assays have been widely used for the detection of a large variety of analytes such as proteins, antibiotics, viruses, and bacteria, because of their high specificity and sensitivity. The enzyme-linked immunosorbent assay (ELISA) is one of the most commonly used analytical methods for analyte quantification. Various commercial ELISA kits have been developed and utilized in clinical diagnostics, environmental monitoring, and food safety applications. However, in many cases, the sensitivity of conventional ELISAs is not sufficient to detect low levels of analytes. Various approaches have been developed to improve the detection sensitivity of immunological assays, including plasmonic ELISA, immuno-polymerase chain reaction (PCR), proximity ligation assay, bio-barcode signal simplification, and single molecule approaches such as single molecule counting.

Small molecule (molecular weight (MW)<5000 Da) detection plays a significant role as biomarkers and in physiological function research, drug discovery, and measurements of harmful substances in environmental and agricultural products. In many cases, small molecules are present at low levels and cannot be detected using conventional approaches. However, because of steric effects caused by their small molecular structure, these small molecules typically cannot be sandwiched by a pair of antibodies used for the detection of proteins.

Thus, there remains a need in the art for high-sensitivity and quantitative detection approaches that can be used to detect and measure the concentration of small molecules, e.g., hormones, peptides, and organic pollutants, alone or in combination with other target analytes.

### SUMMARY OF THE INVENTION

The present invention provides methods and compositions for target analyte (e.g., small molecule) detection and quantification.

In one aspect, the invention features a method of detecting a target analyte in a liquid sample, the method including the steps of: (a) contacting a liquid sample containing or suspected of containing a target analyte with: (i) a plurality of detection probes that specifically bind to the target analyte, and (ii) a plurality of capture probes, the capture probes being linked to one or more immobilized target analytes, wherein the detection probes competitively bind to the target analytes contained in the liquid sample and to the immobilized target analytes; (b) incubating the product of step (a) to allow binding of the detection probes to the target analytes contained in the liquid sample or to the immobilized target analytes; (c) separating at least a portion of the capture probes from the liquid sample; (d) labeling the detection probes that are bound to the immobilized target analytes linked to the capture probes of step (c) with detectable moieties; and (e) detecting the detectable moieties, thereby detecting the target analyte in the liquid sample, wherein the concentration of the target analyte in the liquid sample is inversely proportional to a signal of the detectable moieties. In some embodiments, all or substantially all of the capture probes of step (c) are associated with either zero or one detection probe, wherein a detection probe is associated with a capture probe by binding to a linked immobilized target analyte. In some embodiments, the capture probes are linked to from about 1 to about 1,000,000,000 immobilized target analyte molecules.

In another aspect, the invention features a method of detecting a target analyte in a liquid sample, the method including the steps of: (a) contacting a liquid sample containing or suspected of containing a target analyte with: (i) a plurality of detection probes that specifically bind to the target analyte, and (ii) a plurality of detectable moieties, the detectable moieties being linked to one or more immobilized target analytes, wherein the detection probes competitively bind to the target analytes contained in the liquid sample and to the immobilized target analytes; (b) incubating the product of step (a) to allow binding of the detection probes to the target analytes contained in the liquid sample or to the immobilized target analytes; (c) contacting the product of step (b) with a plurality of capture probes, the capture probes being linked to one or more capture ligands, wherein the capture ligand specifically binds to the detection probe, and incubating to allow capture ligands to bind to detection probes; (d) separating at least a portion of the capture probes from the liquid sample; and (e) detecting the detectable moieties that are associated with the capture probes of step (d), wherein detectable moieties are associated with capture probes by binding of a linked immobilized target analyte to a detection probe that is bound to a capture ligand linked to the capture probe, thereby detecting the target analyte in the liquid sample, wherein the concentration of the target analyte in the liquid sample is inversely proportional to a signal of the detectable moieties. In some embodiments, all or substantially all of the capture probes of step (d) are associated with either zero or one detectable moiety.

In some embodiments of any of the preceding aspects, the target analyte is a small molecule. In some embodiments, the small molecule is an organic compound, an inorganic compound, a steroid, a hormone, a hapten, a biogenic amine, an antibiotic, a mycotoxin, a cyanotoxin, an organic pollutant, a nucleotide, an amino acid, a peptide, a monosaccharide, a nitro compound, a drug residue, a pesticide residue, or a secondary metabolite.

In some embodiments of any of the preceding aspects, the concentration of the target analyte in the liquid sample ranges from about 0 to about 1 mM.

In some embodiments of any of the preceding aspects, the incubating is performed for about 1 min to about 24 h.

In some embodiments of any of the preceding aspects, the detection probe is an antibody, an aptamer, an antibody mimetic, a polypeptide, a nucleic acid, a molecularly-imprinted polymer, a receptor, or a small molecule. In some embodiments, the antibody is a full-length antibody or an antigen-binding antibody fragment. In some embodiments, the full-length antibody is an IgG, IgA, IgD, IgE, or IgM antibody. In some embodiments, the antigen-binding antibody fragment is an scFv, an Fv, a dAb, a Fab, an Fab′, an Fab′₂, an F(ab′)₂, an Fd, an Fv, or an Feb. In some embodiments, the antibody mimetic is an affibody, an affilin, an affimer, an affitin, an alphabody, an anticalin, an avimer, a DARPin, a fynomer, a Kunitz domain peptide, a monobody, or a nanoCLAMP.

In some embodiments of any of the preceding aspects, the capture probes are selected from the group consisting of beads, nanotubes, and polymers. In some embodiments, the beads are paramagnetic beads. In some embodiments, the beads have a size of about 1 μm to about 5 μm.

In some embodiments of any of the preceding aspects, the method includes contacting the liquid sample with about 10,000 to about 2,000,000 capture probes.

In some embodiments of any of the preceding aspects, the detectable moiety is or includes an enzymatic label, a fluorescent label, a radioactive label, or a metal label. In some embodiments, the detectable moiety is or includes an enzymatic label. In some embodiments, the enzymatic label is selected from the group consisting of beta-galactosidase, horseradish peroxidase, glucose oxidase, and alkaline phosphatase.

In some embodiments of any of the preceding aspects, step (d) includes linking the detection probes and the detectable moieties by a non-covalent affinity binding pair, wherein the detection probe is linked to the first member of the non-covalent affinity binding pair, and the detectable moiety is linked to the second member of the non-covalent affinity binding pair. In some embodiments, the non-covalent affinity binding pair is biotin-streptavidin, biotin-avidin, ligand-receptor, antigen-antibody, or antibody binding protein-antibody.

In some embodiments of any of the preceding aspects, the immobilized target analytes are covalently or non-covalently linked to the capture probes.

In some embodiments of any of the preceding aspects, the immobilized target analytes are covalently or non-covalently linked to the detectable moieties.

In some embodiments of any of the preceding aspects, the liquid sample includes a biological sample or an environmental sample. In some embodiments, the biological sample is (i) a body fluid is selected from the group consisting of lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, and spinal fluid, or (ii) a breast tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate. In some embodiments, the biological sample is a body fluid is selected from the group consisting of lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, and spinal fluid. In other embodiments, the biological sample is a breast tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate.

In some embodiments of any of the preceding aspects, the detection of step (e) includes single-molecule detection of the detectable moieties.

In some embodiments of any of the preceding aspects, the detection of step (e) occurs in an array of microwells, wherein the microwells are capable of holding zero or one capture probes. In some embodiments, the array is a QUANTERIX™ single molecule array (Simoa). In some embodiments, the microwells have a volume of about 50 femtoliters.

In other embodiments of any of the preceding aspects, the detection of step (e) occurs in a plurality of water-in-oil droplets. In some embodiments, all or essentially all of the droplets includes zero or one capture probes.

In some embodiments of any of the preceding aspects, the method further includes detecting or measuring a concentration of an additional target analyte in the liquid sample. In some embodiments, the additional target analyte is a small molecule, a protein, a nucleic acid, a polysaccharide, a lipid, a cell, a fatty acid, a therapeutic agent, an organism, a virus, or a small molecule. In some embodiments, step (a) further includes contacting the liquid sample with (i) a plurality of additional detection probes that specifically bind to the additional target analyte; and (ii) a plurality of additional capture probes, the additional capture probes being linked to one or more immobilized additional target analytes, wherein the additional detection probes competitively bind to the additional target analytes contained in the liquid sample and to the immobilized additional target analytes.

In another aspect, the invention features a method of detecting a first target analyte and a second target analyte in a liquid sample, the method including: (a) contacting a liquid sample containing or suspected of containing a first target analyte and/or a second target analyte with: (i) a plurality of first detection probes that specifically bind to the first target analyte; (ii) a plurality of first capture probes, the first capture probes being linked to one or more immobilized first target analytes, wherein the first detection probes competitively bind to the first target analytes contained in the liquid sample and to the immobilized first target analytes; (iii) a plurality of second detection probes that specifically bind to the second target analyte; and (iv) a plurality of second capture probes, the second capture probes being linked to one or more immobilized second target analytes, wherein the second detection probes competitively bind to the second target analytes contained in the liquid sample and to the immobilized second target analytes, wherein the first capture probes and the second capture probes are detectably and distinguishably labeled; (b) incubating the product of step (a) to allow binding of (i) the first detection probes to the first target analytes contained in the liquid sample or to the immobilized first target analytes, and (ii) the second detection probes to the second target analytes contained in the liquid sample or to the immobilized second target analytes; (c) separating at least a portion of the first and the second capture probes from the liquid sample;(d) labeling with detectable moieties (i) the first detection probes bound to the immobilized first target analytes linked to the first capture probes of step (c), and (ii) the second detection probes bound to the immobilized second target analytes linked to the second capture probes of step (c); and (e) detecting the first capture probes of step (d), the second capture probes of step (d), and the detectable moieties associated with the first and second capture probes of step (d), thereby detecting the first target analyte and the second target analyte in the liquid sample, wherein the concentration of the first target analyte in the liquid sample is inversely proportional to a signal of the detectable moieties associated with the first capture probes of step (d), and the concentration of the second target analyte in the liquid sample is inversely proportional to a signal of the detectable moieties associated with the second capture probes of step (d).

In another aspect, the invention features a method of detecting a first target analyte and a second target analyte in a liquid sample, wherein the first target analyte is a small molecule and the second target analyte is a polypeptide, the method including: (a) contacting a liquid sample containing or suspected of containing a first target analyte and/or a second target analyte with: (i) a plurality of first detection probes that specifically bind to the first target analyte; (ii) a plurality of first capture probes, the first capture probes being linked to one or more immobilized first target analytes, wherein the first detection probes competitively bind to the first target analytes contained in the liquid sample and to the immobilized first target analytes; (iii) a plurality of second detection probes that specifically bind to the second target analyte; and (iv) a plurality of second capture probes, the second capture probes being linked to one or more capture ligands, wherein the capture ligand specifically binds to the second target analyte, wherein the first capture probes and the second capture probes are detectably and distinguishably labeled; (b) incubating the product of step (a) to allow binding of (i) the first detection probes to the first target analytes contained in the liquid sample or to the immobilized first target analytes, and (ii) the second target analytes contained in the liquid sample to the second detection probes and to the capture ligands; (c) separating at least a portion of the first and second capture probes from the liquid sample; (d) labeling with detectable moieties (i) the first detection probes bound to the immobilized first target analytes linked to the first capture probes of step (c), and (ii) the second detection probes bound to second target analytes, the second target analytes bound to the capture ligands linked to the second capture probes of step (c); and (e) detecting the first capture probes of step (d), the second capture probes of step (d), and the detectable moieties associated with first and second capture probes of step (d), thereby detecting the first target analyte and the second target analyte in the liquid sample, wherein the concentration of the first target analyte in the liquid sample is inversely proportional to a signal of the detectable moieties associated with the first capture probes of step (d), and the concentration of the second target analyte in the liquid sample is proportional to a signal of the detectable moieties associated with the second capture probes of step (d).

In another aspect, the invention features a composition including: (a) a paramagnetic bead, the paramagnetic bead being linked to one or more immobilized target analytes, wherein the immobilized target analyte is a small molecule; (b) an antibody, the antibody being linked to a biotin moiety; and (c) a beta-galactosidase enzyme, the beta-galactosidase enzyme being linked to a streptavidin moiety, wherein the antibody is bound to one of the immobilized target analytes, and the beta-galactosidase enzyme is bound to the antibody by binding of the biotin moiety to the streptavidin moiety.

In another aspect, the invention features a composition including: (a) a paramagnetic bead, the paramagnetic bead being linked to one or more capture antibodies; (b) a detection antibody; and (c) a beta-galactosidase enzyme linked to an immobilized target ligand, wherein the detection antibody is bound by one of the capture antibodies, and the detection antibody is bound to the immobilized target ligand.

Other features and advantages of the invention will be apparent from the following Detailed Description, the drawings, and the claims.

### BRIEF DESCRIPTION OF THE DRAWINGS

FIG. 1 is a schematic illustration of a competitive single molecule array assay using hapten-bovine serum albumin (BSA)-modified magnetic beads (MBs).

FIG. 2 is a graph showing response curves for the detection of cortisol using a competitive single molecule array assay and a conventional ELISA.

FIG. 3 is a graph showing response curves for the detection of cortisol using different antibodies.

FIG. 4 is a schematic illustration of competitive single molecule array assay using hapten-labeled enzyme.

FIG. 5 is a graph showing response curves for the detection of cortisol using two different assay formats.

FIG. 6 is a schematic illustration of multiplexed detection of different hormones using a single molecule array assay.

FIG. 7 is a graph showing the results of a multiplex assay for the simultaneous detection of cortisol and PGE2.

FIG. 8 is a schematic illustration of multiplexed detection of a protein, IL-6, and a small molecule, cortisol, using a single molecule array assay.

FIG. 9 is a graph showing the results of a multiplex assay for the simultaneous detection of a protein, IL-6, and a small molecule, cortisol.

FIG. 10 shows the chemical structures of cortisol (left panel) and PGE2 (right panel).

FIG. 11 is a schematic illustration of preparation of hapten-β-galactosidase.

FIG. 12 is a graph showing response curves for the detection of cortisol using cortisol-β-galactosidase and two different antibodies.

FIG. 13A is a graph showing response curves for IL-6 using single-plex and multiplex assays.

FIG. 13B is a graph showing response curves for cortisol using single-plex and multiplex assays.

### DETAILED DESCRIPTION OF THE INVENTION

The invention provides methods and compositions for detection or measuring the concentration of a target analyte. The invention is based, at least in part, on the discovery that competitive immunoassays involving single molecule arrays, as described herein, can be used for ultra-sensitive detection of small molecules, including hormones, which are otherwise difficult to detect using antibody pairs in a sandwich format. The methods and assays described herein are unexpectedly significantly more sensitive (approximately 50-fold more sensitive) than known approaches for detection of small molecules. The methods and assays described herein can be multiplexed for simultaneous detection of small molecules and other target analytes (e.g., proteins) in a single sample.

### Definitions

As used herein, the term “about” refers to a value that is within 10% above or below the value being described.

By “target analyte” is meant any atom, molecule, ion, molecular ion, compound, particle, cell, virus, complex, or fragment thereof to be either detected, measured, quantified, or evaluated. A target analyte may be contained in a sample (e.g., a liquid sample (e.g., a biological sample or an environmental sample)). Exemplary target analytes include, without limitation, a small molecule (e.g., an organic compound, a steroid, a hormone, a hapten, a biogenic amine, an antibiotic, a mycotoxin, an organic pollutant, a nucleotide, an amino acid, a monosaccharide, or a secondary metabolite), a protein (including a glycoprotein or a prion), a nucleic acid, a polysaccharide, a lipid, a fatty acid, a cell, a gas, a therapeutic agent, an organism (e.g., a pathogen), or a virus. The target analyte may be naturally occurring or synthetic.

The term “small molecule,” as used herein, means any molecule having a molecular weight of less than 5000 Da. For example, in some embodiments, a small molecule is an organic compound, a steroid, a hormone, a hapten, a biogenic amine, an antibiotic, a mycotoxin, a cyanotoxin, a nitro compound, a drug residue, a pesticide residue, an organic pollutant, a nucleotide, an amino acid, a monosaccharide, or a secondary metabolite.

The terms “nucleic acid” and “polynucleotide,” as used interchangeably herein, refer to at least two covalently linked nucleotide monomers. The term encompasses, e.g., deoxyribonucleic acid (DNA), ribonucleic acid (RNA), hybrids thereof, and mixtures thereof. Nucleotides are typically linked in a nucleic acid by phosphodiester bonds, although the term “nucleic acid” also encompasses nucleic acid analogs having other types of linkages or backbones (e.g., phosphorothioate, phosphoramide, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, and the like). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases.

By “protein” herein is meant at least two covalently linked amino acids, which includes proteins, polypeptides, oligopeptides and peptides. The protein may be made up of naturally occurring amino acids and peptide bonds, or synthetic peptidomimetic structures. Thus “amino acid,” or “peptide residue,” as used herein, means both naturally occurring and synthetic amino acids. For example, homo-phenylalanine, citrulline and norleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. The term “portion” includes any region of a protein, such as a fragment (e.g., a cleavage product or a recombinantly-produced fragment) or an element or domain (e.g., a region of a polypeptide having an activity) that contains fewer amino acids than the full-length or reference polypeptide (e.g., about 5%, 10%, 15%, 20%, 30%, 40%, 50%, 60%, 70%, 80%, 90%, 95%, or 99% fewer amino acids).

The term “detection probe,” as used herein, means any molecule, particle, or the like that is capable of specifically binding to or otherwise specifically associating with a target analyte or another molecule that binds to or otherwise associates with the target analyte (e.g., another detection probe). For example, in some embodiments, a detection probe is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule.

The term “capture probe,” as used herein, means a moiety to which an immobilized target analyte or a capture ligand can be conjugated, captured, attached, bound, or affixed. Detection probes or detectable moieties may bind or otherwise associate with a capture probe in single molecule array assays as described herein. Suitable capture probes include, but are not limited to, beads (e.g., paramagnetic beads), nanotubes, polymers, plates, disks, dipsticks, or the like. In some embodiments, a reaction vessel (e.g., a microwell) is capable of holding zero or one capture probes.

The terms “bead,” “particle,” and “microsphere,” as used interchangeably herein, mean a small discrete particle. Suitable beads include, but are not limited to, paramagnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In some embodiments, spherical beads are used, but it is to be understood that non-spherical or irregularly-shaped beads may be used.

The term “immobilized target analyte,” as used herein, means a target analyte that is conjugated, captured, attached, bound, or affixed to a composition (e.g., a capture probe or a detectable moiety) to prevent or minimize dissociation or loss of the target analyte, but does not require absolute immobility with respect to the composition (e.g., the capture probe or the detectable moiety). The target analyte may be covalently or non-covalently immobilized, e.g., to a capture probe or a detectable moiety. In several embodiments, immobilized target analytes are used in competitive immunoassays as described herein, for example, and may compete with target analytes contained in a sample (e.g., a biological or environmental sample) for binding to a detection probe (e.g., an antibody).

A first moiety “specifically binds” (or grammatical variants thereof) a second moiety if the first moiety (e.g., a detection probe) binds to the second moiety with specificity sufficient to differentiate between the second moiety (e.g., a target analyte or an immobilized target analyte) and other components or contaminants of the test sample. The binding is generally sufficient to remain bound under the conditions of the assay, including wash steps to remove non-specific binding, although in some embodiments, wash steps are not desired; i.e., for detecting low affinity binding partners. In some embodiments, a first moiety specifically binds to a second moiety with an equilibrium dissociation constant (K_{D}) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹²M, 10⁻¹³M, 10⁻¹⁴M, 10⁻¹⁵ M, or lower.

The term “detectable moiety,” as used herein, means a moiety that can produce a detectable signal. For example, in some embodiments, a detectable moiety is or comprises an enzymatic label (e.g., beta-galactosidase, horseradish peroxidase, glucose oxidase, and alkaline phosphatase), a fluorescent label, a radioactive label, or a metal label. In particular embodiments, the detectable moiety is beta-galactosidase.

The term “capture ligand,” as used herein, means a moiety that is capable of specifically binding to or otherwise specifically associating with a detection probe or a target analyte. A capture ligand may be conjugated, captured, attached, bound, or affixed to a capture probe. For example, in some embodiments, a capture ligand is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), an antibody IgG binding protein (e.g., protein A, protein G, protein L, or recombinant protein NG), a polypeptide, a nucleic acid, or a small molecule. For example, in some embodiments, a capture ligand binds to an Fc region of an antibody.

The term “non-covalent affinity binding pair” refers to a pair of moieties that bind and form a non-covalent complex. Exemplary non-covalent affinity binding pairs include, without limitation, biotin-biotin binding protein (e.g., biotin-streptavidin and biotin-avidin), ligand-receptor, antigen-antibody or antigen binding fragment, hapten-anti-hapten, and immunoglobulin (Ig) binding protein-Ig. The members of a non-covalent affinity binding pair may have any suitable binding affinity. For example, the members of an affinity binding pair may bind with an equilibrium dissociation constant (K_{D} or Kd) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or lower.

A “pathogen” is an agent that can cause a disease or illness to its host, including, without limitation, a virus (e.g., a parvovirus (e.g., an adeno-associated virus (AAV)), a retrovirus (e.g., a lentivirus (e.g., human immunodeficiency virus (HIV))), a herpesvirus, an adenovirus, and the like), a bacterium (e.g., E. coli), a protozoon, a fungus, or a prion.

As used herein, “subject” means any animal. In one embodiment, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, lizards, snakes, dogs, fish, hamsters, guinea pigs, rats, mice, and birds).

As used herein, “biomarker” and “marker” interchangeably refer to an analyte (e.g., a small molecule, DNA, RNA, protein, carbohydrate, or glycolipid-based molecular marker), the expression or presence of which in a subject's sample can be detected by methods described herein and is useful, for example, for determining a prognosis, or for monitoring the responsiveness or sensitivity of a mammalian subject to a therapeutic agent.

The term “liquid sample,” as used herein, means a sample that is substantially in liquid form. A liquid sample may include, for example, a biological sample or an environmental sample. It is to be understood that a liquid sample may contain, e.g., particulates or other solid matter.

As used herein, “biological sample” refers to any biological sample obtained from or derived from a subject, including body fluids, body tissue (e.g., tumor tissue), cells, or other sources. Body fluids are, e.g., lymph, whole blood (including fresh or frozen), plasma (including fresh or frozen), serum (including fresh or frozen), a blood fraction containing peripheral blood mononuclear cells, urine, saliva, semen, sweat, lacrimal fluid, synovial fluid, cerebrospinal fluid, feces, mucous, vaginal fluid, and spinal fluid. Samples also include breast tissue, renal tissue, colonic tissue, brain tissue, muscle tissue, synovial tissue, skin, hair follicle, bone marrow, tumor tissue, a tissue lysate or homogenate, or an organ lysate or homogenate. Methods for obtaining tissue biopsies and body fluids from mammals are well known in the art.

By “environmental sample” is meant any sample that is obtained from an environment, e.g., a water sample, soil sample, air sample, extraterrestrial materials, and the like. An environmental sample may contain biological molecules or organisms.

By “array substrate” means any material that can be modified to contain individual discrete sites suitable for the attachment or association of capture probes (e.g., beads) and is amenable to at least one detection method. Suitable array substrates include, but are not limited to, glass and modified or functionalized glass, plastics (e.g., acrylics, polystyrene and copolymers of styrene and other materials, polypropylene, polyethylene, polybutylene, polyurethanes, TEFLON®, and the like), polysaccharides, nylon or nitrocellulose, composite materials, ceramics, and plastic resins, silica or silica-based materials including silicon and modified silicon, carbon, metals, inorganic glasses, plastics, optical fiber bundles, and a variety of other polymers. In general, the substrates allow optical detection and do not appreciably fluoresce.

### Methods

The invention provides methods of detecting a target analyte in a liquid sample. The methods can also involve measuring a concentration of a target analyte. Any of the methods described herein can be used for detecting a target analyte. Detection may be direct or indirect, as described further below.

For example, in one aspect, the invention provides a method of detecting or measuring a concentration of a target analyte in a liquid sample, the method including the steps of: (a) contacting a liquid sample containing or suspected of containing a target analyte with: (i) a plurality of detection probes that specifically bind to the target analyte, and (ii) a plurality of capture probes, the capture probes being linked to one or more immobilized target analytes, wherein the detection probes competitively bind to the target analytes contained in the liquid sample and to the immobilized target analytes; (b) incubating the product of step (a) to allow binding of the detection probes to the target analytes contained in the liquid sample or to the immobilized target analytes; (c) separating at least a portion of the capture probes from the liquid sample; (d) labeling the detection probes that are bound to the immobilized target analytes linked to the capture probes of step (c) with detectable moieties; and (e) detecting the detectable moieties, thereby detecting or measuring the concentration of the target analyte in the liquid sample, wherein the concentration of the target analyte in the liquid sample is inversely proportional to a signal of the detectable moieties. In some embodiments, all or substantially all of the capture probes of step (c) are associated with either zero or one detection probe, wherein a detection probe is associated with a capture probe by binding to a linked immobilized target analyte.

In another aspect, the invention provides a method of detecting or measuring a concentration of a target analyte in a liquid sample, the method including the steps of: (a) contacting a liquid sample containing or suspected of containing a target analyte with: (i) a plurality of detection probes that specifically bind to the target analyte, wherein the detection probes are linked to detectable moieties, and (ii) a plurality of capture probes, the capture probes being linked to one or more immobilized target analytes, wherein the detection probes competitively bind to the target analytes contained in the liquid sample and to the immobilized target analytes; (b) incubating the product of step (a) to allow binding of the detection probes to the target analytes contained in the liquid sample or to the immobilized target analytes; (c) separating at least a portion of the capture probes from the liquid sample; and (d) detecting the detectable moieties, thereby detecting or measuring the concentration of the target analyte in the liquid sample, wherein the concentration of the target analyte in the liquid sample is inversely proportional to a signal of the detectable moieties. In some embodiments, all or substantially all of the capture probes of step (c) are associated with either zero or one detection probe, wherein a detection probe is associated with a capture probe by binding to a linked immobilized target analyte.

In yet another aspect, the invention provides a method of detecting or measuring a concentration of a target analyte in a liquid sample, the method including the steps of: (a) contacting a liquid sample containing or suspected of containing a target analyte with: (i) a plurality of detection probes that specifically bind to the target analyte, and (ii) a plurality of detectable moieties, the detectable moieties being linked to one or more immobilized target analytes, wherein the detection probes competitively bind to the target analytes contained in the liquid sample and to the immobilized target analytes; (b) incubating the product of step (a) to allow binding of the detection probes to the target analytes contained in the liquid sample or to the immobilized target analytes; (c) contacting the product of step (b) with a plurality of capture probes, the capture probes being linked to one or more capture ligands, wherein the capture ligand specifically binds to the detection probe, and incubating to allow capture ligands to bind to detection probes; (d) separating at least a portion of the capture probes from the liquid sample; and (e) detecting the detectable moieties that are associated with the capture probes of step (d), wherein detectable moieties are associated with capture probes by binding of a linked immobilized target analyte to a detection probe that is bound to a capture ligand linked to the capture probe, thereby detecting or measuring the concentration of the target analyte in the liquid sample, wherein the concentration of the target analyte in the liquid sample is inversely proportional to a signal of the detectable moieties. In some embodiments, all or substantially all of the capture probes of step (d) are associated with either zero or one detectable moiety.

In another aspect, provided herein is a method of detecting or measuring a concentration of a target analyte in a liquid sample that includes the following steps: (a) contacting a liquid sample containing or suspected of containing a target analyte with: (i) a plurality of detection probes that specifically bind to the target analyte, (ii) a plurality of detectable moieties, the detectable moieties being linked to one or more immobilized target analytes, wherein the detection probes competitively bind to the target analytes contained in the liquid sample and to the immobilized target analytes; and (iii) a plurality of capture probes capture probes, the capture probes being linked to one or more capture ligands, wherein the capture ligand specifically binds to the detection probe, and incubating to allow capture ligands to bind to detection probes; (b) incubating the product of step (a) to allow binding of the detection probes to the target analytes contained in the liquid sample or to the immobilized target analytes and binding of capture ligands to detection probes; (c) separating at least a portion of the capture probes from the liquid sample; and (d) detecting the detectable moieties that are associated with the capture probes of step (c), wherein detectable moieties are associated with capture probes by binding of a linked immobilized target analyte to a detection probe that is bound to a capture ligand linked to the capture probe, thereby detecting or measuring the concentration of the target analyte in the liquid sample, wherein the concentration of the target analyte in the liquid sample is inversely proportional to a signal of the detectable moieties.

In some embodiments of any of the preceding methods, the capture probes are linked to from about 1 to about 1,000,000,000 immobilized target analyte molecules, e.g., about 1 to about 1,000, about 1 to about 5,000, about 1 to about 10,000, about 1 to about 100,000, about 1 to about 500,000, about 1 to about 1,000,000, about 1 to about 10,000,000, about 1 to about 100,000,000, or about 1 to about 1,000,000,000 immobilized target analytes.

In any of the preceding methods, the target analyte can be a small molecule (e.g., an organic compound, a steroid, a hormone, a hapten, a biogenic amine, an antibiotic, a mycotoxin, a cyanotoxin, a nitro compound, a drug residue, a pesticide residue, an organic pollutant, a nucleotide, an amino acid, a monosaccharide, or a secondary metabolite). The small molecule can be any small molecule described herein (see, e.g., the “Target Analytes” section below). In some embodiments, the target analyte is a biomarker.

In any of the preceding methods, the concentration of the target analyte in the liquid sample can range from about 0 mM to about 5 mM, e.g., about 0 mM to about 5 mM, about 0 mM to about 4 mM, about 0 mM to about 3 mM, about 0 mM to about 2 mM, about 0 mM to about 1 mM, about 0 mM to about 0.5 mM, about 0 mM to about 0.25 mM, about 0.00001 mM to about 5 mM, about 0.00001 mM to about 4 mM, about 0.00001 mM to about 3 mM, about 0.00001 mM to about 2 mM, about 0.00001 mM to about 1 mM, about 0.00001 mM to about 0.5 mM, about 0.00001 mM to about 0.25 mM, about 0.0001 mM to about 5 mM, about 0.0001 mM to about 4 mM, about 0.0001 mM to about 3 mM, about 0.0001 mM to about 2 mM, about 0.0001 mM to about 1 mM, about 0.0001 mM to about 0.5 mM, about 0.0001 mM to about 0.25 mM, about 0.001 mM to about 5 mM, about 0.001 mM to about 4 mM, about 0.001 mM to about 3 mM, about 0.001 mM to about 2 mM, about 0.001 mM to about 1 mM, about 0.001 mM to about 0.5 mM, about 0.001 mM to about 0.25 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.5 mM, about 0.01 mM to about 0.25 mM, about 0.1 mM to about 5 mM, about 0.1 mM to about 4 mM, about 0.1 mM to about 3 mM, about 0.1 mM to about 2 mM, about 0.1 mM to about 1 mM, about 0.1 mM to about 0.5 mM, about 0.1 mM to about 0.25 mM, about 0.2 mM to about 5 mM, about 0.2 mM to about 4 mM, about 0.2 mM to about 3 mM, about 0.2 mM to about 2 mM, about 0.2 mM to about 1 mM, about 0.2mM to about 0.5 mM, about 0.2 mM to about 0.25 mM, about 0.3 mM to about 5 mM, about 0.3 mM to about 4 mM, about 0.3 mM to about 3 mM, about 0.3 mM to about 2 mM, about 0.3 mM to about 1 mM, about 0.3 mM to about 0.5 mM, about 0.4 mM to about 5 mM, about 0.4 mM to about 4 mM, about 0.4 mM to about 3 mM, about 0.4 mM to about 2 mM, about 0.4 mM to about 1 mM, about 0.4 mM to about 0.5 mM, about 0.5 mM to about 5 mM, about 0.5 mM to about 4 mM, about 0.5 mM to about 3 mM, about 0.5 mM to about 2 mM, about 0.5 mM to about 1 mM, about 0.6 mM to about 5 mM, about 0.6 mM to about 4 mM, about 0.6 mM to about 3 mM, about 0.6 mM to about 2 mM, about 0.6 mM to about 1 mM, about 0.7 mM to about 5 mM, about 0.7 mM to about 4 mM, about 0.7 mM to about 3 mM, about 0.7 mM to about 2 mM, about 0.7 mM to about 1 mM, about 0.8 mM to about 5 mM, about 0.8 mM to about 4 mM, about 0.8 mM to about 3 mM, about 0.8 mM to about 2 mM, about 0.8 mM to about 1 mM, about 0.9 mM to about 5 mM, about 0.9 mM to about 4 mM, about 0.9 mM to about 3 mM, about 0.9 mM to about 2 mM, or about 0.9 mM to about 1 mM.

In any of the preceding methods, the incubating can be performed for about 1 min to about 48 h, e.g., about 1 min, about 5 min, about 10 min, about 20 min, about 30 min, about 40 min, about 50 min, about 60 min, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h, about 25 h, about 26 h, about 27 h, about 28 h, about 29 h, about 30 h, about 40 h, or about 48 h.

In any of the preceding methods, the detection probe can be an antibody, an aptamer, an antibody mimetic, a polypeptide, a nucleic acid, a molecularly-imprinted polymer, a receptor, or a small molecule. The antibody may be a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb). The antibody mimetic may be wherein the antibody mimetic is an affibody, an affilin, an affimer, an affitin, an alphabody, an anticalin, an avimer, a DARPin, a fynomer, a Kunitz domain peptide, a monobody, or a nanoCLAMP.

In any of the preceding methods, the capture probe can be a bead, a nanotube, or a polymer. In particular embodiments, the capture probe is a magnetic bead (e.g., a paramagnetic bead). In some embodiments, the beads have a size (e.g., a diameter) of about 0.01 μm to about 10 μm, e.g., about 0.01 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 1.5 μm, about 2 μm, about 2.5 μm, about 3 μm, about 3.5 μm, about 4 μm, about 4.5 μm, about 6 μm, about 6.5 μm, about 7 μm, about 7.5 μm, about 8 μm, about 8.5 μm, about 9 μm, about 9.5 μm, or about 10 μm. In some embodiments, the beads have a size of about 1 μm to about 5 μm, about 1 μm to about 4 μm, about 1 μm to about 3 μm, or about 1 μm to about 2 μm.

Any of the preceding methods may involve contacting the liquid sample with about 1,000 to about 5,000,000 capture probes, e.g., about 1000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, about 1,000,000, about 2,000,000, about 3,000,000, about 4,000,000, or about 5,000,000 capture probes. In some embodiments, the method may involve contacting the liquid sample with about 10,000 to about 5,000,000 capture probes, about 10,000 to about 4,000,000 capture probes, about 10,000 to about 3,000,000 capture probes, about 10,000 to about 2,000,000 capture probes, about 10,000 to about 1,000,000 capture probes, about 10,000 to about 500,000 capture probes, about 10,000 to about 400,000 capture probes, about 10,000 to about 300,000 capture probes, about 10,000 to about 200,000 capture probes, or about 10,000 to about 100,000 capture probes.

In any of the preceding methods, the detectable moiety is or includes an enzymatic label (e.g., beta-galactosidase, horseradish peroxidase, glucose oxidase, and alkaline phosphatase), a fluorescent label, a radioactive label, or a metal label. For example, in some embodiments, an enzymatic label generates a species (for example, a fluorescent product) that is either directly or indirectly detectable optically. In some embodiments, the method includes detecting a product of an enzymatic reaction as an indication of the presence of the enzymatic label. In some embodiments, the product of the enzymatic reaction is detected upon its release from the enzymatic label in a zone around the discrete site where the enzyme and/or target analyte is located (e.g., in a microwell on an array as described herein, e.g., a SIMOA™ array).

In any of the preceding methods, step (d) may include linking the detection probes and the detectable moieties by a non-covalent affinity binding pair, wherein the detection probe is linked to the first member of the non-covalent affinity binding pair, and the detectable moiety is linked to the second member of the non-covalent affinity binding pair. In some embodiments, the non-covalent affinity binding pair is biotin-streptavidin, biotin-avidin, ligand-receptor, antigen-antibody, or antibody binding protein-antibody.

In some embodiments of any of the preceding methods, the immobilized target analytes are covalently or non-covalently linked to the capture probes. The immobilized target analytes may be covalently linked to the capture probes using any suitable conjugation approach known in the art or described herein. The immobilized target analytes may be non-covalently linked to the capture probes, for example, using a non-covalent affinity binding pair.

Any suitable liquid sample may be used in any of the preceding methods. In some embodiments, the liquid sample is or includes a biological sample or an environmental sample. Any suitable biological samples or environmental samples, or derivatives thereof, can be used in the preceding methods, including those described herein.

In some embodiments, detection of step (e) includes single-molecule detection of the detectable moieties. For example, in some embodiments, the detection of step (e) occurs in an array of microwells, wherein the microwells are capable of holding zero or one capture probes. For example, the methods may involve playing a sample on an array substrate, which may contain a plurality of reaction vessels (e.g., microwells). The array may be as described herein or as described, for example, in WO 2014/183096; WO 2010/039179, WO 2009/029073; US 2018/0136203; US 2018/0017552; US 2018/0003703; US 2015/0355182; US 2015/0353997; US 2010/0075355; US 2010/0075439; US 8,846,415; or U.S. Pat. No. 9,482,662, which are incorporated herein by reference in their entirety. In some embodiments, the array is a QUANTERIX™ single molecule array (e.g., SIMOA™). In some embodiments, the microwells have a volume of about 50 femtoliters. In some embodiments, the method further includes sealing the microwells. At least some of the capture probes (e.g., at least some associated with at least one target analyte molecule) may be spatially separated/segregated into a plurality of locations, and at least some of the locations may be addressed/interrogated. A measure of the concentration of target analyte molecules in the fluid sample may be determined based on the information received when addressing the locations. In other embodiments, the detection of step (e) occurs in a plurality of water-in-oil droplets. Any suitable method may be used to make the water-in-oil droplets. In some embodiments, all, essentially all, or a statistically significant proportion of the water-in-oil droplets includes zero or one capture probes.

For example, in some cases, detection or a measure of the concentration may be based at least in part on the number of locations (e.g., microwells) determined to contain a capture probe that is or was associated with at least one detectable moiety. In some embodiments, such as competitive immunoassays for detection of small molecules as described herein, the number of locations determined to contain a capture probe that is or was associated with at least one detectable moiety may be inversely related to the concentration of the target analyte in the sample. In other cases and/or under differing conditions, a measure of the concentration may be based at least in part on an intensity level of at least one signal indicative of the presence of a plurality of target analyte molecules and/or capture probes associated with a target analyte molecule at one or more of the addressed locations. In some embodiments, the number/fraction of locations containing a capture probe but not containing a detectable moiety or a target analyte may also be determined and/or the number/fraction of locations not containing any capture probe may also be determined.

A statistically significant fraction of capture probes that contain at least one detectable moiety or target analyte (or no detectable moieties or target analytes) will typically be able to be reproducibly detected and quantified using a particular system of detection and will typically be above the background noise (e.g., non-specific binding) that is determined when carrying out the assay with a sample that does not contain any target analytes, divided by the total number of objects (or locations) addressed.

Any of the preceding methods may further include detecting or measuring a concentration of one or more additional target analyte(s) in the liquid sample. In some embodiments, the additional target analyte is a small molecule, a protein, a nucleic acid, a polysaccharide, a lipid, a cell, a fatty acid, a therapeutic agent, an organism, or a virus. The additional target analyte may be any suitable target analyte as described herein or known in the art. In some embodiments, step (a) further includes contacting the liquid sample with (i) a plurality of additional detection probes that specifically bind to the additional target analyte; and (ii) a plurality of additional capture probes, the additional capture probes being linked to one or more immobilized additional target analytes, wherein the additional detection probes competitively bind to the additional target analytes contained in the liquid sample and to the immobilized additional target analytes.

The total number of capture probes provided may be between about 10,000 and about 10,000,000, between about 50,000 and about 5,000,000, or between about 100,000 and about 1,000,000. In some cases, the total number of capture probes provided is at least about 10,000, at least about 50,000, at least about 100,000, at least about 1,000,000, at least about 5,000,000, at least about 10,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, or at least about 5,000,000,000.

For example, in another aspect, the invention provides a method of detecting or measuring a concentration of a first target analyte and a concentration of a second target analyte in a liquid sample, the method including: (a) contacting a liquid sample containing or suspected of containing a first target analyte and/or a second target analyte with: (i) a plurality of first detection probes that specifically bind to the first target analyte; (ii) a plurality of first capture probes, the first capture probes being linked to one or more immobilized first target analytes, wherein the first detection probes competitively bind to the first target analytes contained in the liquid sample and to the immobilized first target analytes; (iii) a plurality of second detection probes that specifically bind to the second target analyte; and (iv) a plurality of second capture probes, the second capture probes being linked to one or more immobilized second target analytes, wherein the second detection probes competitively bind to the second target analytes contained in the liquid sample and to the immobilized second target analytes, wherein the first capture probes and the second capture probes are detectably and distinguishably labeled; (b) incubating the product of step (a) to allow binding of (i) the first detection probes to the first target analytes contained in the liquid sample or to the immobilized first target analytes, and (ii) the second detection probes to the second target analytes contained in the liquid sample or to the immobilized second target analytes; (c) separating at least a portion of the first and the second capture probes from the liquid sample; (d) labeling with detectable moieties (i) the first detection probes bound to the immobilized first target analytes linked to the first capture probes of step (c), and (ii) the second detection probes bound to the immobilized second target analytes linked to the second capture probes of step (c); and (e) detecting the first capture probes of step (d), the second capture probes of step (d), and the detectable moieties associated with the first and second capture probes of step (d), thereby detecting or measuring the concentration of the first target analyte and the second target analyte in the liquid sample, wherein the concentration of the first target analyte in the liquid sample is inversely proportional to a signal of the detectable moieties associated with the first capture probes of step (d), and the concentration of the second target analyte in the liquid sample is inversely proportional to a signal of the detectable moieties associated with the second capture probes of step (d).

In another aspect, the invention provides a method of detecting or measuring a concentration of a first target analyte and a concentration of a second target analyte in a liquid sample, wherein the first target analyte is a small molecule and the second target analyte is a polypeptide, the method including: (a) contacting a liquid sample containing or suspected of containing a first target analyte and/or a second target analyte with: (i) a plurality of first detection probes that specifically bind to the first target analyte; (ii) a plurality of first capture probes, the first capture probes being linked to one or more immobilized first target analytes, wherein the first detection probes competitively bind to the first target analytes contained in the liquid sample and to the immobilized first target analytes; (iii) a plurality of second detection probes that specifically bind to the second target analyte; and (iv) a plurality of second capture probes, the second capture probes being linked to one or more capture ligands, wherein the capture ligand specifically binds to the second target analyte, wherein the first capture probes and the second capture probes are detectably and distinguishably labeled; (b) incubating the product of step (a) to allow binding of (i) the first detection probes to the first target analytes contained in the liquid sample or to the immobilized first target analytes, and (ii) the second target analytes contained in the liquid sample to the second detection probes and to the capture ligands; (c) separating at least a portion of the first and second capture probes from the liquid sample; (d) labeling with detectable moieties (i) the first detection probes bound to the immobilized first target analytes linked to the first capture probes of step (c), and (ii) the second detection probes bound to second target analytes, the second target analytes bound to the capture ligands linked to the second capture probes of step (c); and (e) detecting the first capture probes of step (d), the second capture probes of step (d), and the detectable moieties associated with first and second capture probes of step (d), thereby detecting or measuring the concentration of the first target analyte and the second target analyte in the liquid sample, wherein the concentration of the first target analyte in the liquid sample is inversely proportional to a signal of the detectable moieties associated with the first capture probes of step (d), and the concentration of the second target analyte in the liquid sample is proportional to a signal of the detectable moieties associated with the second capture probes of step (d).

A variety of other reagents may be included in the reactions of the preceding methods. These include reagents like salts, neutral proteins, e.g. albumin, detergents, and the like, which may be used to facilitate optimal protein-protein binding and/or reduce non-specific or background interactions. Reagents such as protease inhibitors, nuclease inhibitors, anti-microbial agents, etc., may be used. The mixture of components may be added in any order that provides for the requisite binding. Various blocking and washing steps may be utilized as is known in the art. For example, any of the preceding methods may include one or more (e.g., 1, 2, 3, 4, 5, 6, 7, 8, 9, or 10) wash steps.

Any of the preceding methods may involve providing a prognosis or a diagnosis for a subject based on the concentration of the one or more target analyte(s) in the sample. Any of the preceding methods may involve selecting a therapy for a patient based on the concentration of the one or more target analyte(s) in the sample. Any of the preceding methods may involve treating a subject with a therapy based on the concentration of the one or more target analyte(s) in the sample.

### Detection

Capture probes, detectable moieties, detection probes, and target analytes can be detected and/or quantified, and the detection and/or quantification can be related to the presence and, optionally, the quantity and/or concentration of target analytes in the sample being tested. In some embodiments, a plurality of capture probes, detectable moieties, detection probes, or target analytes may be detected and/or quantified by spatially segregating the plurality of capture probes, detectable moieties, detection probes, or target analytes into a plurality of locations (e.g., in an array). In some embodiments, the plurality of locations comprises a plurality of reaction vessels (e.g., in an array). In some embodiments, a detector may be configured to detect the capture probes, detectable moieties, detection probes, or target analytes in or at a plurality of locations (e.g., an array of reaction vessels). In some embodiments, the capture probes, detectable moieties, detection probes, or target analytes may be able to produce or be made to produce a detectable signal, for example, fluorescence emission, which may aid in the detection of the capture probes, detectable moieties, detection probes, or target analytes. In some cases, the capture probes, detectable moieties, detection probes, or target analytes may be detected using scattering techniques, as described herein.

In some embodiments, non-enzymatic detection methods may be employed. Any suitable non-enzymatic detection method may be used. Non-limiting examples include absorbance, calorimetry (e.g., differential scanning calorimetry (DSC)), circular dichroism, diffraction, electron microscopy (e.g., scanning electron microscopy (SEM), x-ray photoelectron microscopy (XPS)), electron paramagnetic resonance (EPR), electrical transduction methods (e.g., conduction and capacitance), evanescent wave detection, electromagnetic radiation resonance methods (e.g., whispering gallery modes), fluorescence technologies (e.g., fluorescence resonance energy transfer (FRET), time-resolved fluorescence (TRF), fluorescence polarization (FP)), light scattering, luminescent oxygen channeling (LOCI), magnetic transduction effects (e.g., magnetoresistive effect), mass spectroscopy (e.g., matrix assisted laser desorption and ionization (MALDI)), nuclear magnetic resonance (NMR), optical interferometry and other methods based on measuring changes in refractive index, piezoelectric transduction (e.g., quartz crystal microbalance (QCM)), Raman scattering, spectroscopy (e.g., infrared, atomic spectroscopies), scanning probe microscopy (e.g., atomic force microscopy (AFM), scanning tunneling microscopy (STM)), and surface plasmon resonance (SPR).

In some embodiments, indirect detection may be employed. The indirect approach can include, for example, exposing a capture probe, a detectable moiety, a detection probe, or a target analyte to a precursor labeling agent, in which the precursor labeling agent is converted into a labeling agent upon exposure to the capture probe, detectable moiety, detection probe, or target analyte. The labeling agent may comprise a molecule or moiety that can be interrogated and/or detected. The presence or absence of a capture probe, a detectable moiety, a detection probe, or a target analyte at a location may then be determined by determining the presence or absence of a labeling agent at/in the location. For example, the a capture probe, a detectable moiety, a detection probe, or a target analyte may include, be bound to, or associated with an enzymatic label and the precursor labeling agent molecule may be a chromogenic, fluorogenic, or chemiluminescent enzymatic precursor labeling agent molecule which is converted to a chromogenic, fluorogenic, or chemiluminescent product (each an example of a labeling agent) upon exposure to the converting agent. In this instance, the precursor labeling agent may be an enzymatic label, for example, a chromogenic, fluorogenic, or chemiluminescent enzymatic precursor labeling agent, that upon contact with the enzymatic component, is converted into a labeling agent, which is detectable. In some cases, the chromogenic, fluorogenic, or chemiluminescent enzymatic precursor labeling agent is provided in an amount sufficient to contact every location. In some embodiments, an electrochemiluminescent precursor labeling agent is converted to an electrochemiluminescent labeling agent. In some cases, the enzymatic label may comprise beta-galactosidase, horseradish peroxidase, or alkaline phosphatase.

In some embodiments, a plurality of locations may be addressed and/or a plurality of capture probes, detectable moieties, detection probes, or target analytes may be detected substantially simultaneously. Simultaneous addressing/detection can be accomplished by using various techniques, including optical techniques (e.g., using a charge coupled device (CCD) detector, charge-injection device (CID), or complementary-metal-oxide-semiconductor detector (CMOS) detector). Any suitable detector may be used in the methods described herein.

### Target Analytes

As would be appreciated by a person of ordinary skill in the art, a large number of target analytes can be detected and, optionally, quantified using the methods of the invention. Any suitable target analyte can be investigated using the methods of the invention. The target analytes listed below are provided as non-limiting examples. The target analyte may be naturally occurring or synthetic.

In some embodiments, the target analyte is a small molecule. Any suitable small molecule may be detected and, optionally, quantified using the methods of the invention. For example, in some embodiments, the small molecule is an organic compound, an inorganic compound, a steroid (e.g., an androgen/anabolic steroid (e.g., testosterone, 4-hydroxytestosterone, 11-ketotestosterone, boldenone, clostebol, 4-androstenediol, 4-dehydroepiandrosterone (4-DHEA), 5-androstendione, 5-dehydroandrosterone (5-DHA), adrenosterone, adrostenediol, atamestane, cloxotestosterone, quinbolone, silandrone, stanolone, 1-testosterone, nandrolone, or derivatives thereof), an estrogen (e.g., estradiol, 2-hydroxyestradiol, 4-hydroxyestradiol, 4-methoxyestradiol, estrazinol, estrofurate, ethinylestradiol, mestranol, methylestradiol, moxestrol, quinestol, estrone, estriol, or derivatives thereof), a progestogen (e.g., progesterone, quingestrone, retroprogesterone, dydrogesterone, trengestone, hydroxyprogesterone, or derivatives thereof), a corticosteroid (e.g., a glucocorticoid or a mineralcorticoid, including, e.g., cortisol, cortisone, prednisone, prednisolone, methylprednisolone, dexamethasone, betamethasone, triamcinolone, fludrocortisone acetate, deoxycorticosterone acetate, or derivatives thereof), a neurosteroid (e.g., a cholestane (e.g., 25-hydroxycholesterol), an androstane (e.g., 3α-androstanediol, etiocholanediol, and the like), or a pregnane (e.g., 3α-DHP, allopregnanedione, or pregnanedione), a steroid ester, and the like), a hormone (e.g., melatonin, thyroxine, TRH, vasopressin, eicosanoids (e.g., arachidonic acid, lipoxins, thromboxanes, leukotrienes, and prostaglandins (e.g., prostaglandin E2)), steroids as described above, and plant hormones (e.g., abscisic acid, auxin, cytokinin, ethylene, and gibberellin)), a hapten, a biogenic amine (e.g., a monoamine neurotransmitter (e.g., histamine, serotonin, norepinephrine, epinephrine, and dopamine), a trace amine, a thyronamine, tryptamine, trimethylamine, agmatine, adaverine, putrescine, spermine, spermidine, and the like), an antibiotic (e.g., vancomycin, lincosamides (e.g., clindamycin and lincomycin), quinolones (e.g., ciprofloxacin and the like), sulfonamides (e.g., mafenide and the like), macrolides (e.g., azithromycin and clarithromycin), lipopeptide (e.g., daptomycin), dalbavacin, fusidic acid, oxazolidinones (e.g., linezolid), tetracyclines (e.g., minocycline, tetracycline, doxycycline, and the like), mupirocin, oritavancin, tedizolid, telavancin, tigecycline, aminoglycosides (e.g., amikacin, gentamycin, neomycin, kanamycin, tobramycin, and streptomycin), monobactams, carbapenems (e.g., ertapenem, doripenem, imipenem, and meropenem), ceftazidime, tazobactam, penicillins (e.g., penicillin, temocillin, and the like), rifaximin, and cephalosporins (e.g., cefixime, ceftobiprole, and ceftaroline)), a mycotoxin (e.g., aflatoxins, ochratoxins, citrinins, patulins, and fusarium toxins), a cyanotoxin (e.g., microcystin, nodularin, cylindrospermopsin, saxitoxin, neosaxitoxin, and gonyautoxin), an organic pollutant, a nucleotide, an amino acid, a peptide, a monosaccharide (e.g., glucose, fructose, or galactose), a drug residue (e.g., chloramphenicol, clenbuterol, and tylosin), a pesticide residue (e.g., cypermethrin, triazophos, methyl-parathion, fenpropathrin, carbofuran, thiacloprid, chlorothalonil, and carbendazim), or a secondary metabolite (e.g., an alkaloid, a terpenoid, a steroid, a flavonoid, a glycoside, a natural phenol (e.g., resveratrol), a phenazine, a biphenyl, a dibenzofuran, a polyketide, a fatty acid synthase product, a nonribosomal peptide (e.g., vancomycin, ramoplanin, and the like), or a polyphenol).

In some embodiments, the small molecule has a molecular weight of less than about 5000 Da, less than about 4500 Da, less than about 4000 Da, less than about 3500 Da, less than about 3000 Da, less than about 2500 Da, less than about 2000 Da, less than about 1500 Da, less than about 1000 Da, less than about 900 Da, less than about 800 Da, less than about 700 Da, less than about 600 Da, less than about 500 Da, less than about 400 Da, less than about 300 Da, less than about 200 Da, or less than about 100 Da.

In some embodiments, the small molecule has is an organic molecules, including small organic compounds having a molecular weight of more than 100 and less than about 2,500 Da. In some embodiments, the small organic compound may include any suitable functional groups, including an amine, carbonyl, hydroxyl, or carboxyl group, optionally at least two of the functional chemical groups. A small molecule may include cyclical carbon or heterocyclic structures and/or aromatic or polyaromatic structures substituted with one or more of the above functional groups.

Any of the methods described herein may further include detecting and, optionally, quantifying a target analyte that is not a small molecule, for example, in a multiplexed assay (see, e.g., Example 2). For example, in some embodiments, the target analyte that is not a small molecule is, without limitation, a protein (e.g., an antibody, a cytokine (e.g., an interleukin (e.g., IL-1, IL-2, IL-3, IL-4, IL-5, IL-6, IL-7, IL-7, IL-9, IL-10, IL-11, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, or IL-36), a lymphokine, a monokine, an interferon (IFN, e.g., IFN-beta and IFN-gamma), a colony stimulating factor (e.g., CSF, G-CSF, GM-CSF, and the like), a chemokine, a tumor necrosis factor (TNF, including TNF-alpha and TNF-beta), a bone morphogenetic protein (BMP), and the like), a receptor (e.g., an interleukin receptor, a receptor tyrosine kinase, and the like), a ligand, an enzyme (e.g., a polymerase, a cathepsin, a calpain, an aminotransferase (e.g., aspartate aminotransferase (AST) or alanine aminotransferase (ALT)), a protease (e.g., a caspase), a lipase, an oxidoreductase, a kinase, nucleotide cyclases, a transferase, a hydrolase, a lyase, an isomerase, and the like), or a prion), a nucleic acid (e.g., DNA or RNA), a polysaccharide, a lipid, a cell (e.g., a prokaryotic cell (e.g., a bacterium (e.g., E. coli)) or a eukaryotic cell (e.g., a fungal cell or a human cell), including tumor cells), a fatty acid, a glycoprotein, a biomolecule, a therapeutic agent (e.g., an antibody, a fusion protein (e.g., an Fc fusion protein), a cytokine, a soluble receptor, and the like), an organism (e.g., a pathogen), a virus (e.g., a parvovirus (e.g., an adeno-associated virus (AAV)), a retrovirus, a herpesvirus, an adenovirus, a lentivirus, and the like), or a small molecule. In some embodiments, the target analyte may be post-translationally modified (e.g., phosphorylated, methylated, glycosylated, ubiquitinated, and the like).

For example, in some embodiments, the methods may include detecting and, optionally, quantifying, about 1, about 2, about 3, about 4, about 5, about 6, about 7, about 8, about 9, about 10, about 11, about 12, about 14, about 16, about 18, about 20, or more different target analytes.

In some embodiments, the target analyte may be post-translationally modified (e.g., phosphorylated, methylated, glycosylated, ubiquitinated, and the like).

In some embodiments, the target analyte may be a nucleic acid. A nucleic acid may be captured or detected with a complementary nucleic acid fragment (e.g., an oligonucleotide). For example, a detection probe for a nucleic acid target analyte may be or include a complementary oligonucleotide. A detectable moiety (e.g., an enzyme) may bind to a different portion of the nucleic acid target analyte, e.g., using an oligonucleotide that is complementary to a different portion of the nucleic acid target analyte.

### Samples

Any suitable sample may be used in the context of the present invention. For example, in some embodiments, the sample is a liquid sample (e.g., a biological sample or an environmental sample). Exemplary biological samples include, without limitation, body fluids, body tissue (e.g., tumor tissue), cells, or other sources. Exemplary body fluids include, without limitation, e.g., lymph, whole blood (including fresh or frozen), plasma (including fresh or frozen), serum (including fresh or frozen), a blood fraction containing peripheral blood mononuclear cells, urine, saliva, semen, sweat, lacrimal fluid, synovial fluid, cerebrospinal fluid, feces, mucous, vaginal fluid, and spinal fluid. Samples also include breast tissue, renal tissue, colonic tissue, brain tissue, muscle tissue, synovial tissue, skin, hair follicle, bone marrow, tumor tissue, a tissue lysate or homogenate, and an organ lysate or homogenate. Methods for obtaining tissue biopsies and body fluids from mammals are well known in the art. In other embodiments, the sample may be an environmental sample, e.g., a water sample, soil sample, air sample, extraterrestrial materials, or the like.

The volume of the fluid sample analyzed may potentially be any amount within a wide range of volumes, depending on a number of factors such as, for example, the number of capture probes used/available, the number of detection probes, and the like. As non-limiting examples, the sample volume may be about 0.01 μl, about 0.1 μl, about 1 μl, about 5 μl, about 10 μl, about 100 μl, about 1 ml, about 5 ml, about 10 ml, or the like. In some cases, the volume of the fluid sample is between about 0.01 μl and about 10 ml, between about 0.01 μl and about 1 ml, between about 0.01 μl and about 100 μl, or between about 0.1 μl and about 10 μl.

In some embodiments, the fluid sample may be diluted prior to use in a method described herein. For example, in embodiments where the source of an analyte molecule is a body fluid (e.g., blood, plasma, or serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 50-fold, about 100-fold, or greater, prior to use. The sample may be added to a solution comprising the plurality of capture probes or detectable moieties, or the plurality of capture probes or detectable moieties may be added directly to or as a solution to the sample.

### Detection Probes

Any suitable detection probe may be used in the context of the present invention. For example, in some embodiments, the detection probe is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule.

In some embodiments, the detection probe is covalently or non-covalently linked to a detectable moiety or to a member of a non-covalent affinity binding pair.

### Capture Probes and Capture Ligands

Any suitable capture probes can be used in the context of the invention, including, without limitation, beads (e.g., paramagnetic beads), nanotubes, polymers, plates, disks, dipsticks, or the like. Suitable beads include, but are not limited to, paramagnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In preferred embodiments, the bead is a paramagnetic bead. The beads may be substantially spherical or non-spherical.

In some embodiments, immobilized target analytes, detection probes, and/or capture ligands may either be directly synthesized on the capture probes (e.g., beads), or they may be made and then attached after synthesis. In some embodiments, linkers are used to attach the immobilized target analytes, detection probes, and/or capture ligands to the capture probes (e.g., beads), for example, to allow both good attachment, sufficient flexibility to allow good interaction with the target molecule, and to avoid undesirable binding reactions.

As is known in the art, many classes of chemical compounds are currently synthesized on solid supports, such as peptides, organic moieties, and nucleic acids. It is a relatively straightforward matter to adjust the current synthetic techniques to use capture probes (e.g., beads).

In some embodiments, immobilized target analytes, detection probes, and/or capture ligands are obtained or synthesized first, and then covalently attached to the capture probes (e.g., beads). As will be appreciated by those in the art, this will be done depending on the composition of the immobilized target analytes, detection probes, and/or capture ligands and the capture probes (e.g., beads). The functionalization of solid support surfaces such as certain polymers with chemically reactive groups such as thiols, amines, carboxyls, and the like is generally known in the art. Accordingly, “blank” capture probes (e.g., beads) may be used that have surface chemistries that facilitate the attachment of the desired functionality by the user. In certain embodiments, immobilized target analytes, detection probes, and/or capture ligands can be covalently attached to capture probes (e.g., beads) using any suitable chemical reaction, e.g., cycloaddition (e.g., an azide-alkyne Huisgen cycloaddition (e.g., a copper(I)-catalyzed azide-alkyne cycloaddition (CuAAC) or a strain-promoted azide-alkyne cycloaddition (SPAAC))), amide or thioamide bond formation, a pericyclic reaction, a Diels-Alder reaction, sulfonamide bond formation, alcohol or phenol alkylation, a condensation reaction, disulfide bond formation, or a nucleophilic substitution.

In some instances, a composition described herein (e.g., a capture probe, an immobilized target analyte, a detection probe, or a capture ligand) may include a conjugating moiety. A conjugating moiety includes at least one functional group that is capable of undergoing a conjugation reaction, for example, any conjugation reaction described in the preceding paragraph. The conjugation moiety can include, without limitation, a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a thiol, or a thioisocyanate group. In some embodiments, a capture probe may be detectably labeled. For example, in multiplexed assays as described herein, a first population of capture probes may be detectably labeled with a first label, and a second population of capture probes may be detectably labeled with a second label, such that the first population and the second population are distinguishable (also referred to herein as “distinguishably labeled”). Any suitable label can be used. For example, the label may be a reporter dye (e.g., a fluorescent dye, a chromophore, or a phospho), or a mixture thereof). By varying both the composition of the mixture (i.e. the ratio of one dye to another) and the concentration of the dye (leading to differences in signal intensity), matrices of unique tags may be generated. Capture probes (e.g., beads) can be labeled using any suitable approach, for example, by covalently attaching the label (e.g., a dye) to the surface of the capture probes, or alternatively, by entrapping the label (e.g., a dye) within the capture probe. Such dyes may be, for example, covalently attached to the surface of a capture probe (e.g., a bead), for example, using any of the conjugation approaches described above or herein. Suitable dyes for use in the invention include, but are not limited to, ALEXA FLUOR® dyes, CY® dyes, DYLIGHT® dyes, fluorescein, rhodamine, tetramethylrhodamine, eosin, erythrosin, coumarin, methyl-coumarins, pyrene, Malachite green, fluorescent lanthanide complexes, including those of europium and terbium, stilbene, Lucifer Yellow, CASCADE BLUE™, TEXAS RED®, and others known in the art (e.g., as described in The Molecular Probes Handbook, 11^{th} Ed., 2010).

In some embodiments, the methods described herein may involve use of a capture ligand. Any suitable capture ligand can be used in the context of the invention. Exemplary capture ligands include an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a polypeptide, an antibody IgG binding protein (e.g., protein A, protein G, protein L, and recombinant protein A/G), a nucleic acid, or a small molecule. For example, in some embodiments, a capture ligand binds to an Fc region of an antibody. A capture ligand can be covalently or non-covalently attached to a capture probe (e.g., a bead) using any approach known in the art or described herein.

### Detectable Moieties

Any suitable detectable moiety may be used in the context of the invention. For example, a variety of enzymatic labels or colored labels (for example, metallic nanoparticles (e.g., gold nanoparticles), semiconductor nanoparticles, semiconductor nanocrystals (e.g., quantum dots), spectroscopic labels (for example, fluorescent labels), and radioactive labels) may be used in the methods described herein.

Depending upon the particular assay format, the detectable moiety can be indirectly attached, for example, to a target analyte or to a detection probe. In some embodiments, the amount of the detection moiety in a step of a method is inversely proportional to the amount of the target analyte in the sample. The presence of the detectable moiety can be detected using suitable detection systems, for example, optical detectors (for example, intensified CCD cameras), or any other suitable detectors known in the art.

In one embodiment, the detectable moiety is an enzymatic label. In such embodiments, a chromogenic, fluorogenic, or chemiluminescent enzyme substrate may be contacted with the enzyme to produce a detectable product (e.g., a signal). It is understood in the art that chromogenic, fluorogenic, or chemiluminescent enzyme substrates are known or can be made for many different enzymes. Thus, any known chromogenic, fluorogenic, or chemiluminescent enzyme substrate capable of producing a detectable product in a reaction with a particular enzyme can be used in the present invention.

For example, in some embodiments in which the analyte is detected or quantified using a method as described herein in which the enzyme label is β-galactosidase, the enzyme substrate added to the array can be a β-galactosidase substrate such as resorufin-β-D-galactopyranoside or fluorescein di(β-d-galactopyranoside).

### Compositions

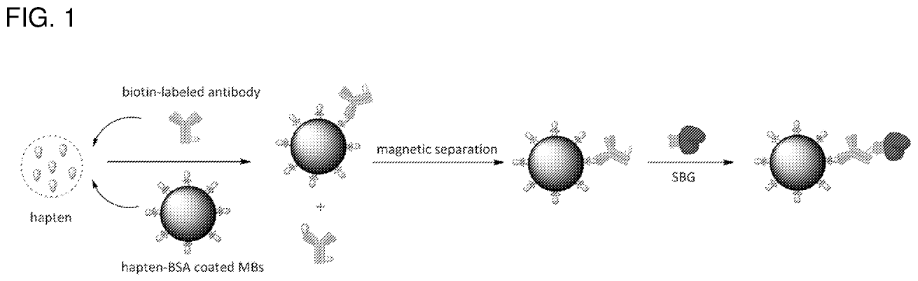

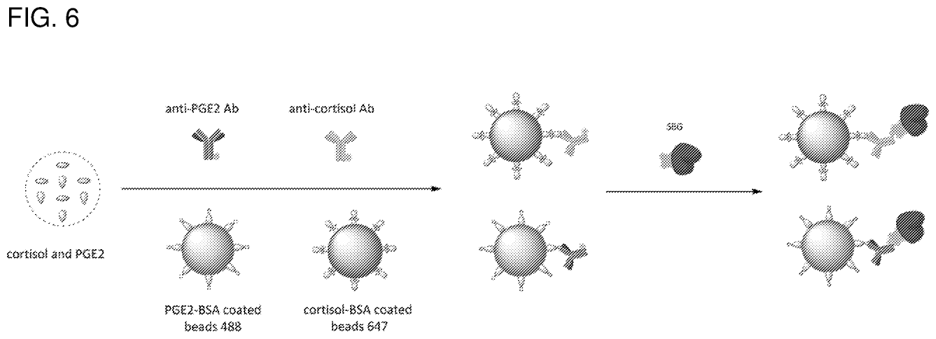

The invention provides compositions which can be used in the detection and, optionally, quantification of target analytes in a sample. See, e.g., FIGS. 1, 4, 6, and 8.

For example, the invention provides a composition that includes: (a) a capture probe (e.g., a paramagnetic bead), the capture probe being linked to one or more immobilized target analytes (e.g., a small molecule); (b) a detection probe (e.g., an antibody),the detection probe being linked to a first member of a non-covalent affinity binding pair (e.g., a biotin moiety); and (c) a detectable moiety (e.g., a beta-galactosidase enzyme), the detectable moiety being linked to a second member of the non-covalent affinity binding pair (e.g., a streptavidin moiety), wherein the detection probe is bound to one of the immobilized target analytes, and the detectable moiety is bound to the detection probe by binding of the first member of the non-covalent affinity binding pair to the second member of the non-covalent affinity binding pair.

In another embodiment, the invention provides a composition that includes: (a) a capture probe (e.g., a paramagnetic bead), the capture probe being linked to one or more capture ligands (e.g., capture antibodies); (b) a detection probe (e.g., a detection antibody); and (c) a detectable moiety (e.g., a beta galactosidase enzyme) linked to an immobilized target ligand, wherein the detection probe is bound by one of the capture ligands, and the detection probe is bound to the immobilized target ligand.

### Arrays

In some embodiments, the methods described herein may utilize a plurality or an array of reaction vessels (e.g., microwells) to determine the presence or concentration of one or more target analytes. An array of reaction vessels allows a fluid sample to be partitioned into a plurality of discrete reaction volumes during one or more steps of a method. In some embodiments, the reaction vessels may all have approximately the same volume. In other embodiments, the reaction vessels may have differing volumes.

The reaction vessels may have any suitable volume. The volume of each individual reaction vessel (e.g., microwell) can range, for example, from attoliters or smaller to nanoliters or larger depending upon the nature of analyte molecules, the detection technique and equipment employed, and the expected concentration of the analyte molecules in the fluid applied to the array for analysis. The size of the reaction vessel may be selected such that at the concentration of interest, between zero and ten capture probes would be statistically expected to be found in each reaction vessel. In a particular embodiment, the volume of the reaction vessel is selected such that at the concentration of interest, either zero or one capture probes would be statistically expected to be found in each reaction vessel.

For example, in some embodiments, the reaction vessels (e.g., microwells) may have a volume between about 1 femtoliter and about 1 picoliter, between about 10 femtoliters and about 100 femtoliters, between about 10 attoliters and about 50 picoliters, between about 1 picoliter and about 50 picoliters, between about 1 femtoliter and about 1 picoliter, between about 30 femtoliters and about 60 femtoliters, or the like. In some embodiments, the reaction vessels (e.g., microwells) have a volume of less than about 1 picoliter, less than about 500 femtoliters, less than about 100 femtoliters, less than about 50 femtoliters, less than about 1 femtoliter, or the like. In some embodiments, the reaction vessels (e.g., microwells) have a volume of about 10 femtoliters, about 20 femtoliters, about 30 femtoliters, about 40 femtoliters, about 50 femtoliters, about 60 femtoliters, about 70 femtoliters, about 80 femtoliters, about 90 femtoliters, or of about 100 femtoliters. In particular embodiments, the reaction vessels (e.g., microwells) have a volume of about 50 femtoliters.

For embodiments employing an array of reaction vessels (e.g., microwells), the number of reaction vessels in the array will depend on the composition and end use of the array. Any suitable number of reaction vessels (e.g., microwells) can be used. Arrays containing from about 2 to many billions of reaction vessels can be made by utilizing a variety of techniques and materials. Increasing the number of reaction vessels in the array can be used to increase the dynamic range of an assay or to allow multiple samples or multiple types of analyte molecules to be assayed in parallel. Generally, the array will comprise between one thousand and one million reaction vessels per sample to be analyzed. In some cases, the array will comprise greater than one million reaction vessels. In some embodiments, the array will comprise between about 1,000 and about 50,000, between about 1,000 and about 1,000,000, between about 1,000 and about 10,000, between about 10,000 and about 100,000, between about 100,000 and about 1,000,000, between about 1,000 and about 100,000, between about 50,000 and about 100,000, between about 20,000 and about 80,000, between about 30,000 and about 70,000, between about 40,000 and about 60,000, or about 50,000, reaction vessels.

The array of reaction vessels may be arranged on a substantially planar surface or, alternatively, in a non-planar three-dimensional arrangement. The reaction vessels may be arrayed in a regular pattern or may be randomly distributed. A preferred embodiment utilizes a regular pattern of sites on a planar structure such that the sites may be addressed in the X-Y coordinate plane. The reaction vessels can be formed in a solid material. As will be appreciated by those in the art, the number of possible materials are very large, and include, but are not limited to, glass and modified or functionalized glass, plastics (including acrylics, polystyrene and copolymers of styrene and other materials, polypropylene, polyethylene, polybutylene, polyurethanes, TEFLON™, and the like), polysaccharides, nylon or nitrocellulose, composite materials, ceramics, and plastic resins, silica or silica-based materials including silicon and modified silicon, carbon, metals, inorganic glasses, plastics, optical fiber bundles, and a variety of other polymers. In general, the substrates allow optical detection and do not appreciably fluoresce.

Individual reaction vessels may contain a binding surface. The binding surface may comprise essentially the entirety or only a portion of the interior surface of the reaction vessel or may be on the surface of another material or object that is confined within the reaction vessel, such as, for example, a bead, or a particle (for example, a micro-particle or a nanoparticle).

In one embodiment, the array of reaction vessels is formed by mating an array of microwells with a sealing component. A microwell may be formed using a variety of techniques known in the art, including, but not limited to, photolithography, stamping techniques, molding techniques, etching techniques, or the like. As will be appreciated by those of the ordinary skill in the art, the technique used will depend on the composition and shape of the supporting material and the size and number of reaction vessels.

For example, in some embodiments, a reaction vessel may be any reaction vessel described in WO 2009/029073, which is incorporated herein by reference in its entirety.

### Kits and Articles of Manufacture

The invention provides kits and articles of manufacture for measuring a concentration of a target analyte (e.g., a small molecule) in a fluid sample. The article or kit may include, for example, a plurality of capture probes (e.g., beads, e.g., paramagnetic beads) and/or an array substrate comprising a plurality of reaction vessels. The reaction vessels may be configured to receive and contain the capture probes. The plurality of capture probes (e.g., beads) may have an average diameter between about 0.1 micrometer and about 100 micrometers and the size of the reaction vessels may be selected such that only either zero or one beads is able to be contained in single reaction vessels. In some cases, the average depth of the reaction vessels is between about 1.0 times and about 1.5 times the average diameter of the beads and the average diameter of the reactions vessels is between about 1.0 times and about 1.9 times the average diameter of the beads. The average volume of the plurality of reaction vessels may be between about 10 attoliters and about 100 picoliters, between about 1 femtoliter and about 1 picoliter, or any desired range. The substrate may comprise any number of reaction vessels, for example, between about 1,000 and about 1,000,000 reaction vessels, between about 10,000 and about 100,000 reaction vessels, or between about 100,000 and about 300,000 reaction vessels, or any other desired range. In certain embodiments, the capture probes (e.g., beads) may have an average diameter between about between about 1 micrometer and about 10 micrometers, between about 1 micrometer and about 5 micrometers, or any range of sizes described herein.

The kits and articles of manufacture described herein may be configured for carrying out any of the methods or assays as described herein, e.g., in the Examples (e.g., Examples 1 and 2).

The plurality of capture probes (e.g., beads) provided may have a variety of properties and parameters, as described herein. For example, the beads may be magnetic. The plurality of beads may comprise a binding surface linked to one or more immobilized target analytes.

In some embodiments, the kit or article may include a detectable moiety that is linked to one or more immobilized target analytes, as described herein.

The plurality of reaction vessels may be formed in any suitable substrate, as described herein or in US 2018/0017552, which is incorporated herein by reference in its entirety. In some embodiments, the plurality of reaction vessels is formed on the end of a fiber optic bundle. The fiber optic bundle may be prepared (e.g., etched) according to methods known to those of ordinary skill in the art and/or methods described in US 2018/0017552. In other embodiments, the plurality of reactions vessels is formed in a plate or similar substantially planar material (e.g., using lithography or other known techniques). Exemplary suitable materials are described herein. The kit may include any of the array substrates or reaction vessels as described herein.

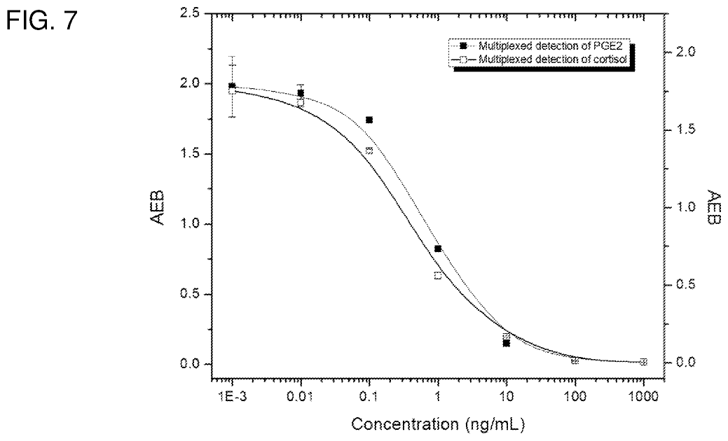

The kit or article may comprise any number of additional components, some of which are described in detail herein. In some cases, the article or kit may further comprise a sealing component configured for sealing the plurality of reaction vessels. In certain embodiments, the plurality of reaction vessels may be formed upon the mating of at least a portion of a sealing component and at least a portion of the second substrate, as shown in FIGS. 7A-7F of US 2018/0017552. As another example, the kit may also provide solutions for carrying out an assay method as described herein. Non-limiting example of solutions include solutions containing one or more types of detection probes, capture probes, or enzymatic label substrates. In some cases, the article or kit may comprise at least one type of control capture probe (e.g., a bead).

In some embodiments, the kit may include instructions for use of components described herein. That is, the kit can include a description of use of the capture probes (e.g., beads) and reaction vessels, for example, for use with a system to determine a measure of the concentration of target analyte(s) in a fluid sample. As used herein, “instructions” can define a component of instruction and/or promotion, and typically involve written instructions on or associated with packaging of the invention. Instructions also can include any oral or electronic instructions provided in any manner such that a user of the kit will clearly recognize that the instructions are to be associated with the kit. Additionally, the kit may include other components depending on the specific application, as described herein.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Materials and Methods

### 1.1 Materials

Cortisol solution, hydrocortisone 3-(O-carboxymethyl)oxime (cortisol-3-CMO), β-galactosidase (G5635), anti-mouse IgG (Fc specific) antibody (M4280), 2-(N-morpholino)ethanesulfonic acid (MES), AMICON® Ultra 0.5 mL centrifugal filters, TWEEN® 20, and bovine serum albumin (A7030) were purchased from Sigma Aldrich (St. Louis, Mo.). 1-ethyl-3-(3-dimethylaminopropyl)carbodiimide hydrochloride (EDC), N-hydroxysulfosuccinimide (sulfo-NHS), NHS-PEG4-Biotin, and anti-cortisol monoclonal antibody (clone: F4P1A3) were purchased from Thermo Fisher (Waltham, Mass.). Cortisol-BSA conjugate was purchased from Fitzgerald Industries International (Acton, Mass.). Prostaglandin E2 (PGE2) was purchased from Enzo Life Sciences (Farmingdale, N.Y.). Anti-PGE2 monoclonal antibody (414013) was purchased from Cayman Chemical (Ann Arbor, Mich.). Anti-cortisol monoclonal antibody (clone: XM210) was purchased from GeneTex (Irvine, Calif.). Detection antibodies were biotinylated using NHS-PEG4-Biotin according to a previously reported method (Wu et al. Analyst 140:6277-6282, 2015). Recombinant human IL-6 protein (206 IL), IL-6 capture (MAB206) and detection (BAF206) antibodies were purchased from R&D Systems (Minneapolis, Minn.). The SIMOA HD-1 ANALYZER™ and Homebrew assay kits were purchased from Quanterix Corporation (Lexington, Mass.). Homebrew kits include carboxyl-functionalized paramagnetic beads, 159 nM streptavidin-β-galactosidase (SβG) concentrate, 100 μM resorufin β-D-galactopyranoside (RGP), and diluents (Bead Diluent, Sample Diluent, and SβG Diluent).

### 1.2 Preparation of Hapten-β-Galactosidase Conjugates

Cortisol-3-CMO and PGE2 were respectively dissolved in methanol to a final concentration of 1 mg/mL. EDC and sulfo-NHS were reconstituted in MES buffer (50 mM, pH 6.2) to a final concentration of 10 mg/mL. 25 μL of each of the EDC and sulfo-NHS were added to 50 μL of hapten solution. The mixture was vortexed and incubated at room temperature for 1 h. Cortisol-3-CMO-NHS ester or PGE2-NHS ester was added to 100 μL of 2 mg/mL β-galactosidase in 1× phosphate buffered saline (PBS) with a final molar ratio of 50:1. The solution was mixed and incubated at room temperature for 30 min. Excess NHS esters were removed by using AMICON® centrifugation filtration with a cutoff value of 100 kDa. The concentration of hapten-β-galactosidase conjugates (E^{1%} [280 nm]=20.93) were measured on a NANODROP™ 2000 Spectrophotometer (Thermo Fisher). Hapten-β-galactosidase conjugates were stored in SβG Diluent at 4° C.

### 1.3 Preparation of Capture Beads

Carboxylated 2.7 μm paramagnetic beads (˜4×10⁸), non-encoded for single-plex assays and dye-encoded (488 nm and 647 nm) for multiplex assays, were washed three times with 600 μL of bead wash buffer (0.1% TWEEN® 20 in 1× PBS, pH 7.4) and twice with 400 μL of MES buffer (50 mM MES, pH 6.2). 1 mg/mL EDC in MES buffer was freshly prepared and 200 μL was added to the beads and mixed well. The beads were activated on a shaker for 30 min. After activation, the beads were washed once with 200 μL of MES buffer. 200 μL of 10 mg/mL BSA or BSA-cortisol conjugate in MES buffer were added to the activated beads. The beads were incubated at room temperature with shaking for 2 h, and then washed three times with bead wash buffer. The BSA-cortisol conjugate coated beads were stored in 200 μL of bead storage buffer (50 mM Tris-HCl with 1% BSA, 1% TRITON® 100 and 0.15% PROCLIN® 300, pH 7.8) at 4° C. for further use. The BSA coated beads were resuspended in 200 μL of 1× PBS, and then 50 μL of 2.8 mM PGE2-NHS ester was added. The beads were vortexed, incubated with shaking for 30 min, and then washed five times with bead wash buffer to remove unreacted PGE2-NHS ester. The BSA-PGE2 coated beads were finally resuspended in 200 μL of bead storage buffer and stored at 4° C. In addition, anti-mouse IgG and anti-IL-6 capture beads were prepared according to a previously published method (Cohen et al. J. Immunol. Methods 452:20-25, 2018). A two-plex single molecule array assay was developed for simultaneous detection of IL-6 and cortisol. 488 nm and 647 nm dye-encoded beads were coated with IL-6 capture antibody and BSA-cortisol conjugate, respectively. The beads were counted using a Beckman-Coulter MULTISIZER™.

### 1.4 Preparation of Reagents and Assay Setup for Single Molecule Array Assay

A two-step assay configuration was chosen when hapten-BSA-coated beads were used. Specifically, hapten-BSA-coated capture beads were diluted in Bead Diluent (Quanterix) to a concentration of 5,000 beads/μL. Biotinylated detector antibodies were diluted in Detector Diluent (Quanterix) to the desired concentration (1 pM for anti-cortisol antibodies, and 1 nM for anti-PGE2 antibodies). SβG concentrate was diluted to 200 pM in SβG Diluent (Quanterix). Cortisol and PGE2 standards were serially diluted to desired concentrations in Sample Diluent. The reagents including beads, detector, and SβG were placed in plastic bottles (Quanterix). The samples were loaded onto a 96-well plate (Quanterix). All reagents (capture beads, detector antibodies, SβG, enzyme substrate RGP, Wash Buffer 1, Wash Buffer 2, and SIMOA™ Sealing Oil) were purchased from Quanterix and loaded onto the SIMOA HD-1 ANALYZER™ (Quanterix) based on the manufacturer's instructions. 100 μL of bead solution was pipetted into a reaction cuvette. The beads were pelleted with a magnet and the supernatant was removed. 100 μL of sample and 25 μL of detector antibody were then added and incubated for 30 min. The beads were then pelleted again and the supernatant was removed. Following a series of washes, 100 μL of SβG was added and incubated. The beads were washed, resuspended in RGP solution, and loaded onto the array. The array was then sealed with oil and imaged. Images of the arrays were analyzed and AEB (average enzyme per bead) values were calculated by the software in the SIMOA HD-1 ANALYZER™.

A two-step assay configuration was used for simultaneous detection of IL-6 and cortisol. BSA-cortisol coated beads were mixed with anti-IL6 capture beads (1:1 in number). The mixed beads were diluted to a concentration of 10,000 beads/μL. A mixture of detection antibodies, 10 pM for anti-cortisol antibody (XM210) and 1 nM for anti-IL6 detector, was prepared in Detector Diluent. A mixture of cortisol and IL-6 standards was serially diluted to desired concentrations. All the other conditions were the same as the single-plex assay.

A one-step assay configuration was chosen when hapten-β-galactosidase was used as the competitor. The concentration of anti-mouse beads was 5,000 beads/μL. Unlabeled detector antibodies were diluted in Detector Diluent to the desired concentration (6 pM for anti-cortisol antibodies, and 6 nM for anti-PGE2 antibodies). Hapten-β-galactosidase concentrate was diluted to 1.2 nM in SβG Diluent. 100 μL of bead solution was pipetted into a reaction cuvette. The beads were pelleted with a magnet and the supernatant was removed. Then 100 μL of sample, 25 μL of detector antibody, and 25 μL of hapten-β-galactosidase were added and incubated for 30 min. All the other steps were the same as that of a two-step assay.

### 1.5 ELISA for Cortisol

The high-binding 96-well microtiter plate was coated with BSA-cortisol conjugate in PBS (1 μg/mL, 100 μL/well) overnight at 4° C. After washing, the plate was blocked with 1% BSA in PBS (200 μL/well) for 1 h at room temperature and subsequently washed. Next, biotinylated anti-cortisol (10 nM, 10 μL/well) in PBS and serial concentrations of cortisol standard (0, 0.001, 0.01, 0.1, 1, 10, 100 and 1,000 ng/mL,100 μL/well) prepared in Sample Diluent were added to the wells. The immunoreaction was allowed to proceed for 1 h. After washing, the plate was incubated with streptavidin-horseradish peroxidase (HRP((2 nM, 100 μL/well) for 30 min. After another washing, 3,3′,5,5′-tetramethylbenzidine (TMB) substrate (100 μL/well) was added and the plate was incubated for 15 min. Next, 50 μL of 1 mol/L hydrochloric acid was added to each well to stop the enzymatic reaction. Finally, the optical density (OD) was recorded at 450 nm on a Tecan Infinite M200 Plate Reader.

### 1.6 Data Analysis

Standard curves were obtained by plotting the signal responses (AEB and OD) against the logarithm of analyte concentrations using Origin software (Origin 9.5). The 4-parameter logistic equation y=A₂+(A₁−A₂)/[1+(x/x₀)^{P}] was used for curve fitting in the whole concentration range, where A₁ is the maximum signal without analyte, A₂ is the minimum signal at infinite concentration, p is the curve slope at the inflection point, and x₀ is the IC50 (analyte concentration causing a 50% inhibition of the maximum response). The lower the IC50 is, the higher the sensitivity is. The limit of detection (LOD) was calculated as three standard deviations (SDs) above the background. All measurements were performed in triplicate.

### Example 2: Competitive Immunoassays for the Detection of Small Molecules using Single Molecule Arrays

Recently, an ultra-sensitive detection method known as digital ELISA has been developed for detection of proteins using single molecule array assays (e.g., SIMOA™). In this approach, proteins are first captured on antibody-modified paramagnetic beads. Excess beads are used compared to the number of target analyte molecules to ensure that there is either zero or one target protein molecule bound per bead. A second biotinylated detection antibody binds to the captured target protein molecule, and the immunocomplex is then labeled with streptavidin-β-galactosidase (SβG). The enzyme-labeled beads are resuspended in a fluorogenic substrate solution, resorufin-β-D-galactopyranoside (RGP), and loaded onto an array of microwells (50 fL) in which each well is able to hold only one bead. The wells are sealed with oil and the fluorescent product generated by the enzymatic reaction is confined within the microwells, ensuring high local fluorescence intensity that can be easily detected by a charge coupled device (CCD) camera. The whole assay process can be conducted in an automatic fashion with the SIMOA HD-1 ANALYZER® (Quanterix Corporation). The SIMOA™ technique has been previously utilized for ultra-sensitive detection of DNA and microRNAs, in which DNA probes instead of antibodies were used to capture the target molecules. While large molecules, such as proteins and nucleic acids have been measured using the SIMOA™ technique, small molecules have been a challenge because they are too small to bind to the two different binding agents used for sandwich-type assays.

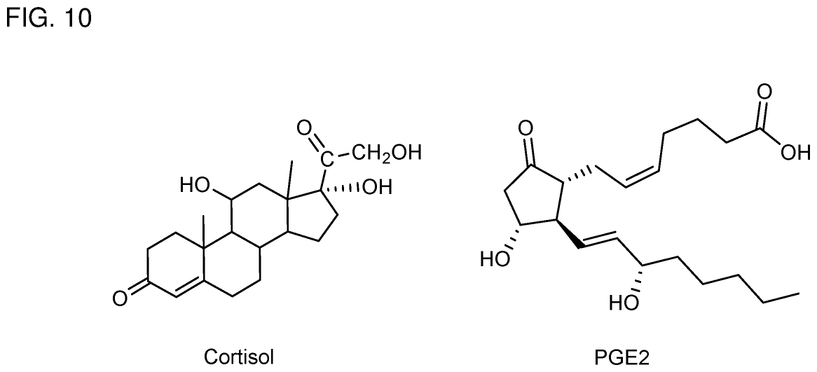

To solve this problem, we developed a competitive single molecule array (e.g., SIMOA™) assay using only one monoclonal antibody for the detection of small molecules. In this study, we present, to our knowledge, the first example of small molecule detection using the single molecule array (e.g., SIMOAT™) technique. Two small molecular compounds (haptens), cortisol and prostaglandin E2 (PGE2), were selected as model targets (FIG. 10). As shown in FIG. 1, BSA-hapten conjugate-modified magnetic beads (MBs) were employed as capture probes, while biotin-labeled antibodies were used as detection probes for immunological recognition of the target. After incubation and magnetic separation, SβG was added to label the beads for the single molecule array (e.g., SIMOA™) assay. In addition, multiplexed detection of IL-6 and cortisol was performed by using dye-encoded MBs, which, to the best of our knowledge, is the first example in which both proteins and small molecules are simultaneously detected in one sample using an immunoassay.

### 2.1 Competitive Single Molecule Array Assay

We applied single molecule array assay to small molecule detection by developing a bead-based competitive protocol. As shown in FIG. 1, biofunctionalized MBs and free target molecules in the sample competitively bind to biotin-labeled antibodies. After incubation and magnetic separation, the supernatant containing unbound antibodies was removed. The beads labeled with detection probes were then labeled with an enzyme, SβG, via biotin-streptavidin interaction and detected by enzymatic readout on the SIMOA™ platform. The signal from the assay was measured in units of average enzyme per bead (AEB), as previously described (Rissin et al. Anal. Chem. 83:2279-2285, 2011). Because of competitive inhibition, an increase in the number of target molecules leads to a decrease in the number of biotinylated antibody molecules that bind to the MBs, resulting in lower signal. Thus, the signal intensity (AEB) is inversely proportional to the analyte concentration.

2.1.1 Comparison with Cconventional ELISA

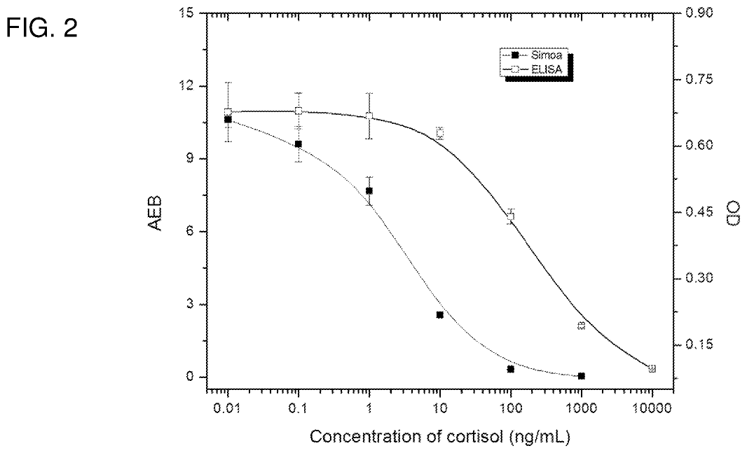

Competitive ELISAs have been used for detection of various small molecules. We compared the analytical performance of the competitive single molecule array assay with conventional ELISA for the detection of cortisol. BSA-cortisol conjugates were immobilized either on a microtiter plate or MBs, and the same detection antibodies were used. Cortisol molecules in the sample and immobilized BSA-cortisol conjugates competitively bound to biotinylated detection probes. After washing, streptavidin-labeled enzyme was added for signal development. As shown in FIG. 2, the shape of the response curve for the single molecule array assay was similar to that of the ELISA. With increasing cortisol concentration, the signal intensity (AEB for single molecule array and OD for ELISA) decreased proportionally. The response curve for the single molecule array assay was left-shifted to lower concentration compared with the ELISA. The half maximal inhibitory concentration (IC50), an important parameter to evaluate the sensitivity of a competitive immunoassay, was 3.20 ng/mL for the single molecule array assay (Table 1). In contrast, the IC50 for the conventional ELISA was 170.23 ng/mL, which was 50-fold higher than that of the single molecule array assay (Table 1). This result demonstrates that a competitive single molecule array assay as described herein is much more sensitive than a conventional ELISA.

TABLE 1
Analytical performance of different assays for cortisol.

| | | | | Anti-mouse IgG |
|---|---|---|---|---|
| | Kd: | ELISA | BSA-cortisol MBs | antibody MBs |
| Antibody | nM | IC50: ng/mL | IC50: ng/mL | IC50: ng/mL |
| XM210 | 0.59 | 18.60 | 0.42 | 1.06 |
| F4P1A3 | 1.0 | 170.23 | 3.20 | 4.07 |

2.1.2 The Effects of Antibody Affinity

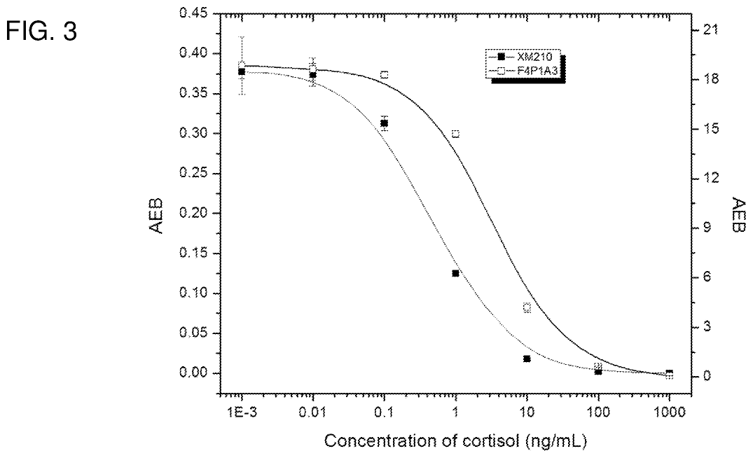

Antibody affinity can have a major impact on immunoassay performance. The lower the dissociation constant of the antibody, the higher the affinity to its ligand. To test the effect of antibody affinity on single molecule array and ELISA performance, we selected two monoclonal anti-cortisol antibodies with dissociation constants of 0.59 nM and 1.0 nM. The antibody with the lower Kd, XM210, showed a higher affinity towards cortisol than the antibody with the higher Kd, F4P1A3 (Table 1). When using the single molecule array assay, the IC50 values were 0.42 ng/mL for the higher affinity antibody and 3.20 ng/mL for the lower affinity antibody. FIG. 3 shows the response curves of the single molecule array assays. When using a conventional ELISA, the IC50 values were 18.60 ng/mL for the higher affinity antibody and 170.23 ng/mL for the lower affinity antibody. This indicates the sensitivity of competitive immunoassays is dependent on the affinity of detection antibodies but that single molecule array consistently improves the analytical sensitivity over ELISA.

2.1.3 Comparison Between Two Assay Formats

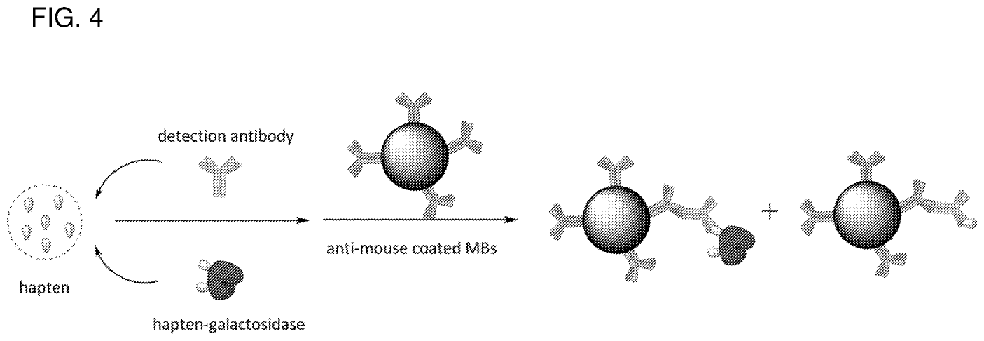

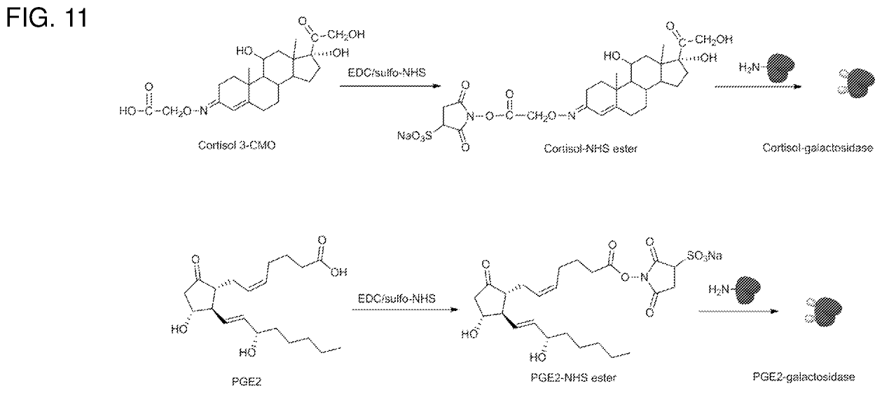

Another assay protocol was developed using hapten-labeled β-galactosidase as the competitor. Hapten-labeled enzyme was prepared through a reaction between activated carboxyl groups of the hapten and amino groups of β-galactosidase (FIG. 11). As shown in FIG. 4, labeled enzymes compete with free target hapten molecules in binding to the detection antibodies. The Fc region of the detection antibody was then specifically captured by anti-mouse IgG antibody modified MBs. Because of competitive inhibition, an increase in the number of target molecules leads to a decrease in the number of enzyme molecules that bind to the MBs, resulting in lower signal. In this assay format, instead of conjugating the detection antibodies directly to the MBs, we conjugated anti-mouse IgG antibodies, which capture the Fc region of the detection antibodies, to the MBs. Using this format, the detection antibody concentration can be easily controlled and a lower detection antibody concentration can be used. If the detection antibody was directly conjugated to the MBs, the concentration would be more difficult to control and be substantially higher, in the nM range, which may compromise the assay sensitivity.

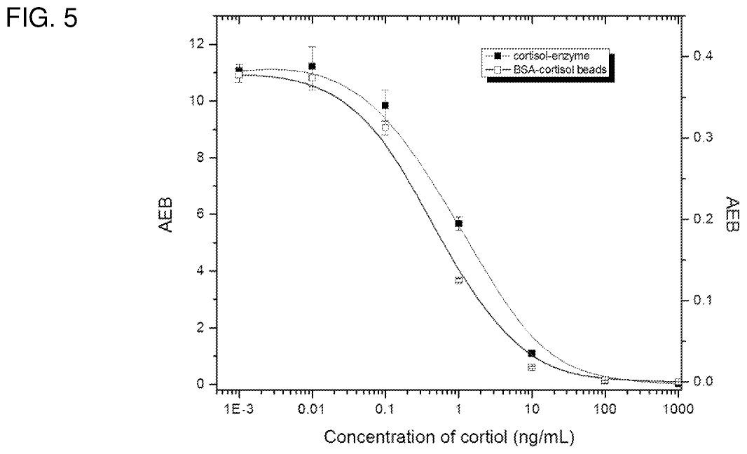

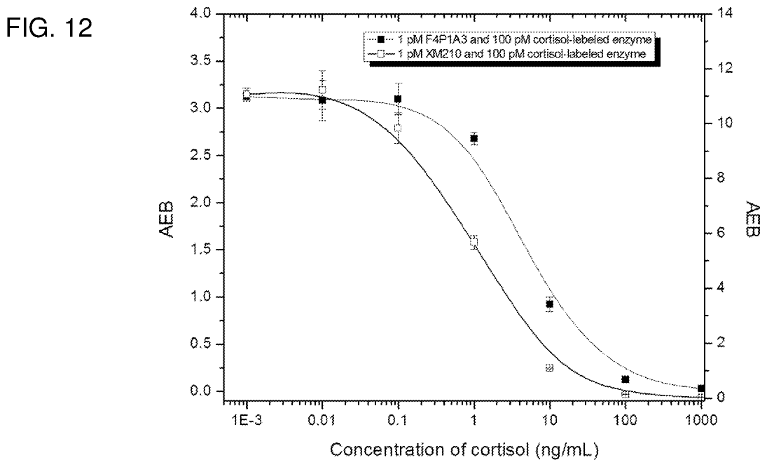

We also compared this new assay format to that described earlier in the text (see FIG. 1). As shown in FIG. 5, the response curve was slightly right-shifted when hapten-labeled enzyme was used as the competitor, indicating the sensitivity was lower than when using hapten-modified MBs. The IC50s were 1.06 and 4.07 ng/mL for XM210 and F4P1A3, respectively (FIG. 12). This also demonstrated that the sensitivity was dependent on the affinity of detection antibodies. Both single molecule array assays were still much more sensitive than conventional ELISAs (Table 1).

### 2.2 Multiplexed Detection of Small Molecules

Simultaneous detection of multiple different target analytes in a single sample increases throughput and requires less sample volume compared to detection of each target individually. We developed a two-plex single molecule array assay for simultaneous detection of two widely-investigated hormones, PGE2 and cortisol. To enable multiplexing, we used paramagnetic beads labeled with different fluorescent dyes to produce distinct bead subpopulations (Rissin et al. Lab on a Chip 13:2902-2911, 2013; Rivnak et al. J. lmmunol. Methods 424:20-27, 2015). Each subpopulation of beads was modified with BSA-hapten conjugates for a specific hormone. For the detection of PGE2, MBs were first modified with BSA, and then PGE2-NHS ester was added, which specifically reacted with amine groups on BSA molecules. Cortisol and PGE2 were simultaneously detected following the procedures described in the Example 1 (FIG. 7). IC50s for cortisol and PGE2 were 0.46 and 0.7 ng/mL, respectively. The sensitivity was comparable with that of single-plex assays, which are 0.42 ng/mL for cortisol and 0.37 ng/mL for PGE2, demonstrating that multiplexing does not compromise sensitivity.

### 2.3 Multiplexed Detection of Proteins and Small Molecules

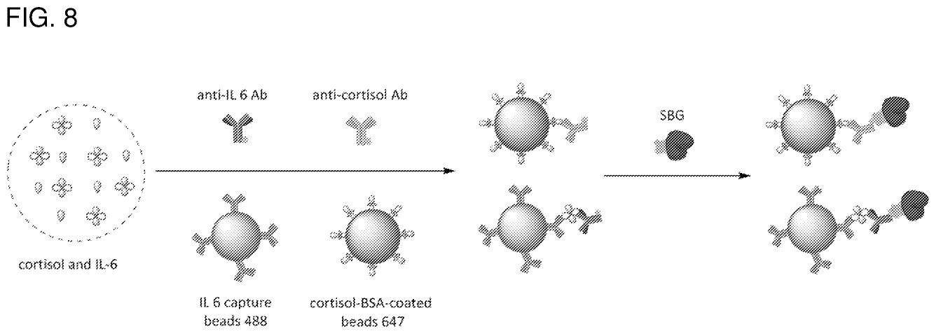

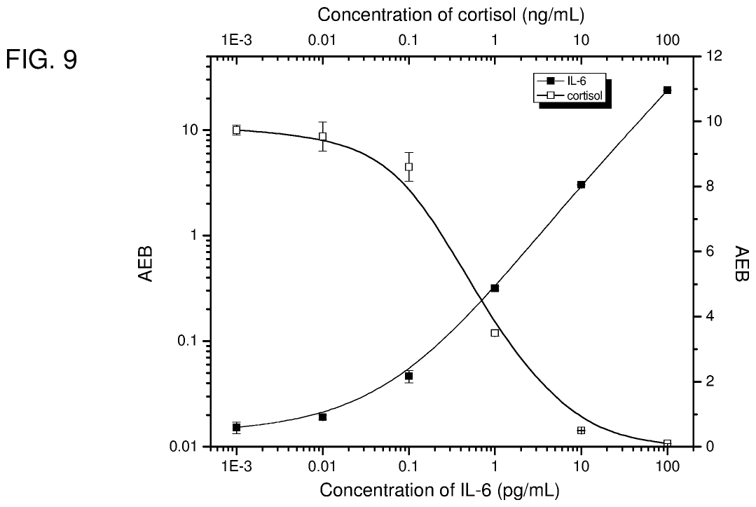

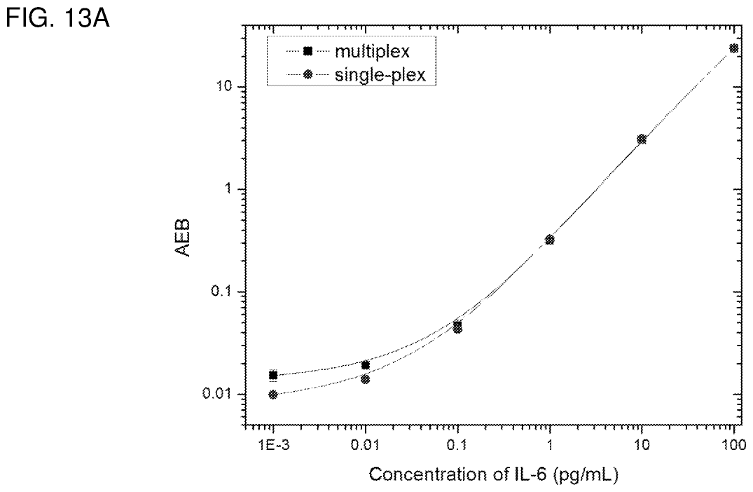

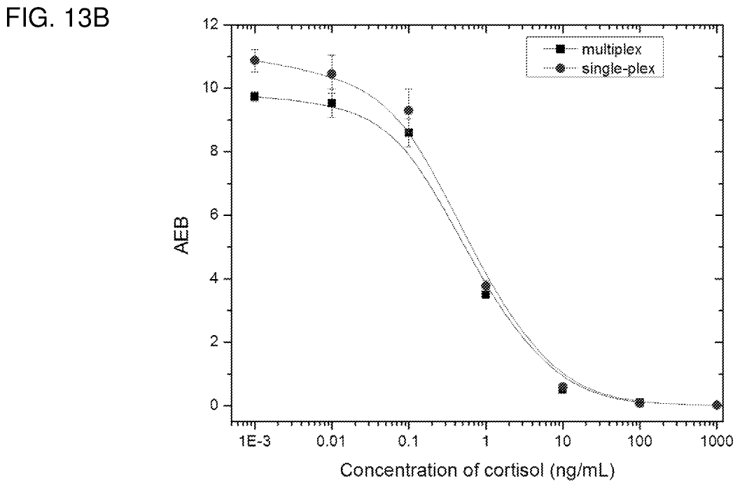

Biological fluids such as blood, serum, and saliva usually contain a large variety of molecules including proteins, amino acids, peptides, inorganic salts, and hormones. Many of these molecules can serve as potential biomarkers of disease and therefore, it is desirable to simultaneously detect different classes of biomarkers in one sample. Multiplexed single molecule immunoassays have been developed to simultaneously detect several proteins in serum samples (Rissin et al. supra; Rivnak et al. supra). Here we developed a multiplex single molecule array assay for simultaneous detection of proteins and hormones in a single sample. IL-6 and cortisol were chosen as model targets since they both are present in biological fluids such as blood, serum, and saliva. As shown in FIG. 8, 488 nm and 647 nm dye-encoded MBs were coated with IL-6 capture antibody and BSA-cortisol conjugate, respectively. A mixture of beads, a sample containing IL-6 and cortisol, and a mixture of biotinylated detection antibodies were incubated together. IL-6 was captured on the beads and then sandwiched by the detection probes. BSA-cortisol coated MBs competed with cortisol in the sample for binding with anti-cortisol antibodies. Only a portion of anti-cortisol antibodies were captured by the beads because of competitive inhibition. After magnetic separation and washing, SβG was added to label the captured detection probes for single molecule array assays. The response curves are shown in FIG. 9. The LOD for IL-6 was 0.020 pg/mL (0.007 pg/mL for single-plex assay), and the IC50 for cortisol was 0.57 ng/mL (0.54 ng/mL for single-plex assay). Only a small deviation from background was observed at low concentrations (FIGS. 13A and 13B). These results indicate that cortisol and IL-6 can be simultaneously detected in a multiplex single molecule array assay with high sensitivity and specificity.

### 2.4 Conclusions

Competitive single molecule array assays (e.g., SIMOA™) have been developed for the detection of small molecules with good reproducibility and high sensitivity. The sensitivity of the proposed method was approximately 50 times higher than that of the conventional ELISA for cortisol. Detection sensitivity depends in part on the affinity of antibodies used. In addition, multiple small molecular analytes can be simultaneously detected with high sensitivity by using dye-encoded MBs. Proteins and small molecules in one sample can also be simultaneously detected with high sensitivity. Multiplexing capabilities enable measurements of several analytes simultaneously and thus enhance efficiency. The approaches described herein provide a platform which can be utilized for the detection of other small molecular analytes in a diverse range of areas, such as environmental monitoring, food safety, and medical diagnostics.

What is claimed is:

1. A method of detecting a target analyte in a liquid sample, the method comprising the steps of:
(a) contacting a liquid sample containing or suspected of containing a target analyte with:
(i) a plurality of detection probes that specifically bind to the target analyte, and
(ii) a plurality of capture probes, the capture probes being linked to one or more immobilized target analytes, wherein the detection probes competitively bind to the target analytes contained in the liquid sample and to the immobilized target analytes;
(b) incubating the product of step (a) to allow binding of the detection probes to the target analytes contained in the liquid sample or to the immobilized target analytes;
(c) separating at least a portion of the capture probes from the liquid sample;
(d) labeling the detection probes that are bound to the immobilized target analytes linked to the capture probes of step (c) with detectable moieties; and
(e) detecting the detectable moieties, thereby detecting the target analyte in the liquid sample, wherein the concentration of the target analyte in the liquid sample is inversely proportional to a signal of the detectable moieties.

2. The method of claim 1, wherein all or substantially all of the capture probes of step (c) are associated with either zero or one detection probe, wherein a detection probe is associated with a capture probe by binding to a linked immobilized target analyte.

3. The method of claim 1 or 2, wherein the capture probes are linked to from about 1 to about 1,000,000,000 immobilized target analyte molecules.

4. A method of detecting a target analyte in a liquid sample, the method comprising the steps of:
(a) contacting a liquid sample containing or suspected of containing a target analyte with:
(i) a plurality of detection probes that specifically bind to the target analyte, and
(ii) a plurality of detectable moieties, the detectable moieties being linked to one or more immobilized target analytes, wherein the detection probes competitively bind to the target analytes contained in the liquid sample and to the immobilized target analytes;
(b) incubating the product of step (a) to allow binding of the detection probes to the target analytes contained in the liquid sample or to the immobilized target analytes;
(c) contacting the product of step (b) with a plurality of capture probes, the capture probes being linked to one or more capture ligands, wherein the capture ligand specifically binds to the detection probe, and incubating to allow capture ligands to bind to detection probes;
(d) separating at least a portion of the capture probes from the liquid sample; and
(e) detecting the detectable moieties that are associated with the capture probes of step (d), wherein detectable moieties are associated with capture probes by binding of a linked immobilized target analyte to a detection probe that is bound to a capture ligand linked to the capture probe, thereby detecting the target analyte in the liquid sample, wherein the concentration of the target analyte in the liquid sample is inversely proportional to a signal of the detectable moieties.

5. The method of claim 4, wherein all or substantially all of the capture probes of step (d) are associated with either zero or one detectable moiety.

6. The method of any one of claims 1-5, wherein the target analyte is a small molecule.

7. The method of claim 6, wherein the small molecule is an organic compound, an inorganic compound, a steroid, a hormone, a hapten, a biogenic amine, an antibiotic, a mycotoxin, a cyanotoxin, an organic pollutant, a nucleotide, an amino acid, a peptide, a monosaccharide, a nitro compound, a drug residue, a pesticide residue, or a secondary metabolite.

8. The method of any one of claims 1-6, wherein the concentration of the target analyte in the liquid sample ranges from about 0 to about 1 mM.

9. The method of any one of claims 1-8, wherein the incubating is performed for about 1 min to about 24 h.

10. The method of any one of claims 1-9, wherein the detection probe is an antibody, an aptamer, an antibody mimetic, a polypeptide, a nucleic acid, a molecularly-imprinted polymer, a receptor, or a small molecule.

11. The method of claim 10, wherein the antibody is a full-length antibody or an antigen-binding antibody fragment.

12. The method of claim 11, wherein the full-length antibody is an IgG, IgA, IgD, IgE, or IgM antibody.

13. The method of claim 11, wherein the antigen-binding antibody fragment is an scFv, an Fv, a dAb, a Fab, an Fab′, an Fab′₂, an F(ab′)₂, an Fd, an Fv, or an Feb.

14. The method of claim 10, wherein the antibody mimetic is an affibody, an affilin, an affimer, an affitin, an alphabody, an anticalin, an avimer, a DARPin, a fynomer, a Kunitz domain peptide, a monobody, or a nanoCLAMP.

15. The method of any one of claims 1-14, wherein the capture probes are selected from the group consisting of beads, nanotubes, and polymers.

16. The method of claim 15, wherein the beads are paramagnetic beads.

17. The method of claim 15 or 16, wherein the beads have a size of about 1 μm to about 5 μm.

18. The method of any one of claims 1-17, wherein the method comprises contacting the liquid sample with about 10,000 to about 2,000,000 capture probes.

19. The method of any one of claims 1-18, wherein the detectable moiety is or comprises an enzymatic label, a fluorescent label, a radioactive label, or a metal label.

20. The method of claim 19, wherein the detectable moiety is or comprises an enzymatic label.

21. The method of claim 20, wherein the enzymatic label is selected from the group consisting of beta-galactosidase, horseradish peroxidase, glucose oxidase, and alkaline phosphatase.

22. The method of any one of claim 1-3 or 6-21, wherein step (d) comprises linking the detection probes and the detectable moieties by a non-covalent affinity binding pair, wherein the detection probe is linked to the first member of the non-covalent affinity binding pair, and the detectable moiety is linked to the second member of the non-covalent affinity binding pair.

23. The method of claim 22, wherein the non-covalent affinity binding pair is biotin-streptavidin, biotin-avidin, ligand-receptor, antigen-antibody, or antibody binding protein-antibody.

24. The method of any one of claim 1-3 or 6-23, wherein the immobilized target analytes are covalently or non-covalently linked to the capture probes.

25. The method of any one of claims 4-23, wherein the immobilized target analytes are covalently or non-covalently linked to the detectable moieties.

26. The method of any one of claims 1-25, wherein the liquid sample comprises a biological sample or an environmental sample.

27. The method of claim 26, wherein the biological sample is (i) a body fluid selected from the group consisting of lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, and spinal fluid, or (ii) a breast tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate.

28. The method of any one of claims 1-27, wherein the detection of step (e) comprises single-molecule detection of the detectable moieties.

29. The method of any one of claims 1-28, wherein the detection of step (e) occurs in an array of microwells, wherein the microwells are capable of holding zero or one capture probes.

30. The method of claim 29, wherein the array is a QUANTERIX™ single molecule array (Simoa).

31. The method of claim 29 or 30, wherein the microwells have a volume of about 50 femtoliters.

32. The method of any one of claims 1-28, wherein the detection of step (e) occurs in a plurality of water-in-oil droplets.

33. The method of claim 32, wherein essentially all of the droplets includes zero or one capture probes.

34. The method of any one of claims 1-33, further comprising detecting or measuring a concentration of an additional target analyte in the liquid sample.

35. The method of claim 34, wherein the additional target analyte is a small molecule, a protein, a nucleic acid, a polysaccharide, a lipid, a cell, a fatty acid, a therapeutic agent, an organism, a virus, or a small molecule.

36. The method of claim 34 or 35, wherein step (a) further comprises contacting the liquid sample with
(i) a plurality of additional detection probes that specifically bind to the additional target analyte;
and (ii) a plurality of additional capture probes, the additional capture probes being linked to one or more immobilized additional target analytes,
wherein the additional detection probes competitively bind to the additional target analytes contained in the liquid sample and to the immobilized additional target analytes.

37. A method of detecting a first target analyte and a second target analyte in a liquid sample, the method comprising:
(a) contacting a liquid sample containing or suspected of containing a first target analyte and/or a second target analyte with:
(i) a plurality of first detection probes that specifically bind to the first target analyte;
(ii) a plurality of first capture probes, the first capture probes being linked to one or more immobilized first target analytes, wherein the first detection probes competitively bind to the first target analytes contained in the liquid sample and to the immobilized first target analytes;
(iii) a plurality of second detection probes that specifically bind to the second target analyte; and
(iv) a plurality of second capture probes, the second capture probes being linked to one or more immobilized second target analytes, wherein the second detection probes competitively bind to the second target analytes contained in the liquid sample and to the immobilized second target analytes,
wherein the first capture probes and the second capture probes are detectably and distinguishably labeled;
(b) incubating the product of step (a) to allow binding of (i) the first detection probes to the first target analytes contained in the liquid sample or to the immobilized first target analytes, and (ii) the second detection probes to the second target analytes contained in the liquid sample or to the immobilized second target analytes;
(c) separating at least a portion of the first and the second capture probes from the liquid sample;
(d) labeling with detectable moieties (i) the first detection probes bound to the immobilized first target analytes linked to the first capture probes of step (c), and (ii) the second detection probes bound to the immobilized second target analytes linked to the second capture probes of step (c); and
(e) detecting the first capture probes of step (d), the second capture probes of step (d), and the detectable moieties associated with the first and second capture probes of step (d), thereby detecting the first target analyte and the second target analyte in the liquid sample, wherein the concentration of the first target analyte in the liquid sample is inversely proportional to a signal of the detectable moieties associated with the first capture probes of step (d), and the concentration of the second target analyte in the liquid sample is inversely proportional to a signal of the detectable moieties associated with the second capture probes of step (d).

38. A method of detecting a first target analyte and a second target analyte in a liquid sample, wherein the first target analyte is a small molecule and the second target analyte is a polypeptide, the method comprising:
(a) contacting a liquid sample containing or suspected of containing a first target analyte and/or a second target analyte with:
(i) a plurality of first detection probes that specifically bind to the first target analyte;
(ii) a plurality of first capture probes, the first capture probes being linked to one or more immobilized first target analytes, wherein the first detection probes competitively bind to the first target analytes contained in the liquid sample and to the immobilized first target analytes;
(iii) a plurality of second detection probes that specifically bind to the second target analyte; and
(iv) a plurality of second capture probes, the second capture probes being linked to one or more capture ligands, wherein the capture ligand specifically binds to the second target analyte,
wherein the first capture probes and the second capture probes are detectably and distinguishably labeled;
(b) incubating the product of step (a) to allow binding of (i) the first detection probes to the first target analytes contained in the liquid sample or to the immobilized first target analytes, and (ii) the second target analytes contained in the liquid sample to the second detection probes and to the capture ligands;
(c) separating at least a portion of the first and second capture probes from the liquid sample;
(d) labeling with detectable moieties (i) the first detection probes bound to the immobilized first target analytes linked to the first capture probes of step (c), and (ii) the second detection probes bound to second target analytes, the second target analytes bound to the capture ligands linked to the second capture probes of step (c); and
(e) detecting the first capture probes of step (d), the second capture probes of step (d), and the detectable moieties associated with first and second capture probes of step (d), thereby detecting the first target analyte and the second target analyte in the liquid sample, wherein the concentration of the first target analyte in the liquid sample is inversely proportional to a signal of the detectable moieties associated with the first capture probes of step (d), and the concentration of the second target analyte in the liquid sample is proportional to a signal of the detectable moieties associated with the second capture probes of step (d).

39. A composition comprising:
(a) a paramagnetic bead, the paramagnetic bead being linked to one or more immobilized target analytes, wherein the immobilized target analyte is a small molecule;
(b) an antibody, the antibody being linked to a biotin moiety; and
(c) a beta-galactosidase enzyme, the beta-galactosidase enzyme being linked to a streptavidin moiety,
wherein the antibody is bound to one of the immobilized target analytes, and the beta-galactosidase enzyme is bound to the antibody by binding of the biotin moiety to the streptavidin moiety.

40. A composition comprising:
(a) a paramagnetic bead, the paramagnetic bead being linked to one or more capture antibodies;
(b) a detection antibody; and
(c) a beta-galactosidase enzyme linked to an immobilized target ligand,
wherein the detection antibody is bound by one of the capture antibodies, and the detection antibody is bound to the immobilized target ligand.
